(19) United States
(12) Patent Application Publication
(10) Pub. No.: US 2023/0248424 A1
(43) Pub. Date: Aug. 10, 2023

(54) ENDOSCOPIC TREATMENT DEVICE

(71) Applicant: OLYMPUS MEDICAL SYSTEMS CORP., Tokyo (JP)

(72) Inventors: Yusuke SHIOTA, Machida-shi (JP); Chika MIYAJIMA, Hachioji-shi (JP); Hiromasa KATO, Tokyo (JP); Junichi KOGISO, Hachioji-shi (JP); Yohei YOSHIDA, Fuchu-shi (JP); Yasuhito SUGA, Musashino-shi (JP); Yasuo FUNAKOSHI, Hachioji-shi (JP); Yui KAWANISHI, Koganei-shi (JP)

(73) Assignee: OLYMPUS MEDICAL SYSTEMS CORP., Tokyo (JP)

(21) Appl. No.: 18/107,606

(22) Filed: Feb. 9, 2023

Related U.S. Application Data
(60) Provisional application No. 63/308,535, filed on Feb. 10, 2022.

(51) Int. Cl. A61B 18/14

(52) U.S. Cl. CPC A61B 18/1492; A61B 2018/1475; A61B 2218/003; A61B 2218/005; A61B 2018/00601

(57) ABSTRACT

An endoscopic treatment device includes a hollow tube; an electrode; and an insulative support member. In a radial direction relative to a longitudinal axis of the hollow tube, the insulative support member is radially outward of the electrode and the hollow tube is radially outward of the insulative support member.

### CROSS-REFERENCE TO RELATED APPLICATIONS

The present disclosure claims priority on U.S. Provisional Application No. 63/308,535, filed Feb. 10, 2022. The disclosure of the prior applications is hereby incorporated by reference herein in its entirety.

### TECHNICAL FIELD

The present disclosure relates to an endoscopic treatment device.

### BACKGROUND

Conventionally, in an endoscopic treatment such as the ESD (Endoscopic Submucosal Dissection), as shown in Japanese Unexamined Patent Application, First Publication No. 2013-111308, the endoscopic treatment devices for incision and dissection and endoscopic treatment devices for hemostasis such as the high-frequency knives are used. According to the ESD, the surgeon injects locally into the lesion in the luminal cavity of the gastrointestinal tract to swell the lesion and surrounding living tissue, and then performs incision and resection treatment using the endoscopic treatment device for incision and resection. The surgeon incises the living tissue around the lesion using the endoscopic treatment device. The surgeon turns up the incised living tissue and peels off the incised lesion little by little.

### SUMMARY

An endoscopic treatment device includes a hollow tube; an electrode; and an insulative support member. In a radial direction relative to a longitudinal axis of the hollow tube, the insulative support member is radially outward of the electrode and the hollow tube is radially outward of the insulative support member.

An endoscopic treatment device includes a hollow tube; an electrode; and an insulative support member. In a radial direction relative to a longitudinal axis of the hollow tube, the insulative support member is radially outward of the electrode. The insulative support member is movable relative to the electrode in a direction of the longitudinal axis of the hollow tube.

An endoscopic treatment method includes spraying a fluid from a treatment tool onto a mucous membrane; and while spraying the fluid from the treatment tool, moving the treatment tool toward a lesion portion of the mucous membrane to turn up the mucous membrane, and incising a submucosa layer.

### DESCRIPTION OF DRAWINGS

FIG. 1 is an overall view showing an endoscopic treatment system according to a first embodiment.

FIG. 2 is an overall view showing a treatment device of the endoscopic treatment system.

FIG. 3 is a perspective view showing a distal-end portion of the treatment device.

FIG. 4 is a cross-sectional view showing the distal-end portion of the treatment device in which a rod is protruded.

FIG. 5 is a cross-sectional view showing the distal-end portion of the treatment device in which a support portion is protruded.

FIG. 6 is a cross-sectional view showing the distal-end portion of the treatment device in which the rod and the support portion is protruded.

FIG. 7 is a view showing an incision step by the endoscopic treatment system.

FIG. 8 is a view showing a dissection step by the endoscopic treatment system.

FIG. 9 is a perspective view showing a modification example of a forceps of the treatment device.

FIG. 10 is a perspective view showing the modification example.

FIG. 11 is a perspective view showing the modification example.

FIG. 12 is a view showing a modification example of a distal-end portion of the forceps.

FIG. 13 is a view showing another modification example of the distal-end portion of the forceps.

FIG. 14 is a perspective view showing another modification example of the forceps.

FIG. 15 is a perspective view showing the modification example.

FIG. 16 is a perspective view showing another modification example of the forceps.

FIG. 17 is a view showing a dissection step using the forceps according to the modification example.

FIG. 18 is a view showing a modification example of an operation portion of the treatment device.

FIG. 19 is a view showing the modification example.

FIG. 20 is an overall view showing a treatment device of an endoscopic treatment system according to a second embodiment.

FIG. 21 is a perspective view showing a distal-end portion of the treatment device.

FIG. 22 is a front view showing the distal-end portion of the treatment device.

FIG. 23 is a cross-sectional view showing the distal-end portion of the treatment device in which a rod is accommodated.

FIG. 24 is a cross-sectional view showing the distal-end portion of the treatment device from which the rod is protruded.

FIG. 25 is a front view showing a distal-end portion of the treatment device as another aspect.

FIG. 26 is a front view showing a distal-end portion of the treatment device as another aspect.

FIG. 27 is a front view showing a distal-end portion of the treatment device as another aspect.

FIG. 28 is a front view showing a distal-end portion of the treatment device as another aspect.

FIG. 29 is a front view showing a distal-end portion of the treatment device as another aspect.

FIG. 30 is a view showing a dissection step by the endoscopic treatment system.

FIG. 31 is a view showing the dissection step.

FIG. 32 is a view showing the dissection step.

FIG. 33 is a view showing the dissection step.

FIG. 34 is a view showing a hemostasis step.

FIG. 35 is a view showing the hemostasis step.

FIG. 36 is a view showing the hemostasis step.

FIG. 37 is a view showing the hemostasis step.

FIG. 38 is a cross-sectional view showing a modification example of a distal-end tip of the treatment device.

FIG. 39 is a view showing a dissection step using the treatment device according to the modification example.

FIG. 40 is a view showing a hemostasis step using the treatment device according to the modification example.

FIG. 41 is an overall view showing a treatment device of an endoscopic treatment system according to a third embodiment.

FIG. 42 is a perspective view showing a distal-end portion of the treatment device.

FIG. 43 is a side view showing the distal-end portion of the treatment device.

FIG. 44 is a cross-sectional view showing the distal-end portion of the treatment device.

FIG. 45 is a cross-sectional view showing the distal-end portion of the treatment device.

FIG. 46 is a view showing a local-injection step by the endoscopic treatment system.

FIG. 47 is a view showing the local-injection step.

FIG. 48 is a view showing the local-injection step.

FIG. 49 is a perspective view showing a distal-end portion of a treatment device of an endoscopic treatment system according to a fourth embodiment.

FIG. 50 is a cross-sectional view showing a distal-end portion of the treatment device.

FIG. 51 is a view showing a local-injection step by the endoscopic treatment system.

FIG. 52 is a view showing the local-injection step.

FIG. 53 is a view showing the local-injection step.

FIG. 54 is a perspective view showing a distal-end portion of a treatment device of an endoscopic treatment system according to a fifth embodiment.

### DETAILED DESCRIPTION

### First Embodiment

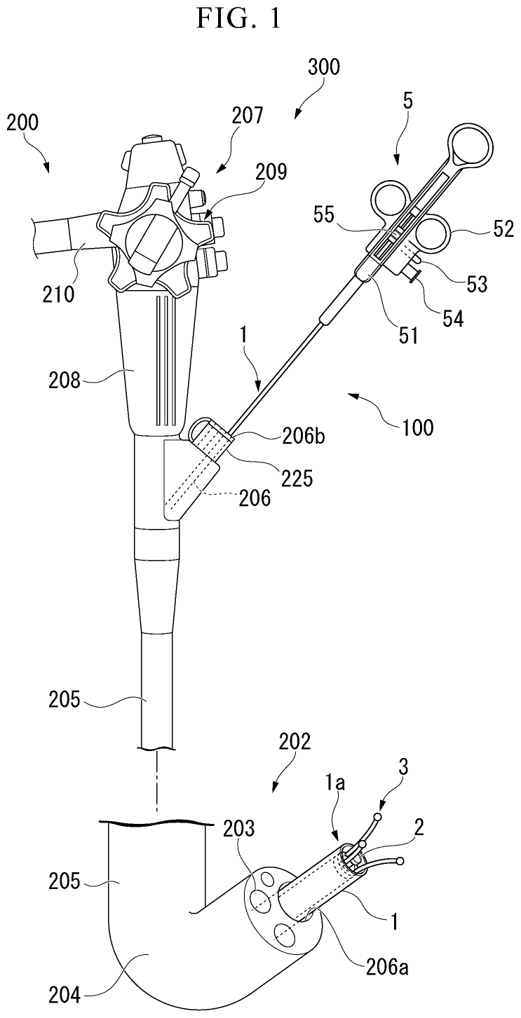

An endoscopic treatment system 300 according to a first embodiment of the present disclosure will be described with reference from FIG. 1 to FIG. 8. FIG. 1 is an overall view of the endoscopic treatment system 300 according to the present embodiment.

### [Endoscopic Treatment System 300]

As shown in FIG. 1, the endoscopic treatment system 300 includes an endoscope 200 and a treatment device 100. The treatment device 100 is inserted into the endoscope 200 to be used.

### [Endoscope 200]

The endoscope 200 is a conventional flexible endoscope, and the endoscope 200 includes an insertion portion 202 that is inserted into the body from a distal end thereof and an operation portion 207 that is attached to a proximal end of the insertion portion 202.

The insertion portion 200 includes an imaging portion 203, a bending portion 204, and a flexible portion 205. The imaging portion 203, the bending portion 204, and the flexible portion 205 are arranged in this sequence from the distal end of the insertion portion 202. Inside the insertion portion 202, a channel 206 for inserting the treatment device 100 thereinto is provided. At the distal end of the insertion portion 202, a distal-end opening portion 206a of the channel 206 is provided.

The imaging portion 203 includes an imaging element such as a CCD or a CMOS and the like, and is capable of imaging a portion as a treatment target. The imaging portion 203 can image a rod 2 of the treatment device 100 while the treatment device 100 protrudes from the distal-end opening portion 206a of the channel 206.

The bending portion 204 is bent following the operations of the operation portion 207 by the operator. The flexible portion 205 is a tubular portion having the flexibility.

The operation portion 207 is connected to the flexible portion 205. The operation portion 207 includes a grip 208, an input portion 209, a proximal-end opening portion (forceps port) 206b of the channel 206, and a universal cord 210. The grip 208 is a portion being grasped by the operator. The input portion 209 receives the operation input for operating the bending portion 204 to be bent. A forceps plug 225 is attached to the proximal-end opening portion (forceps port) 206b to prevent the leakage of bodily fluids. The universal cord 210 outputs the image captured by the imaging portion 203 to the outside. The universal cord 210 is connected to a display device such as a liquid crystal display or the like via an image processing device including a processor and the like.

### [Treatment Device 100]

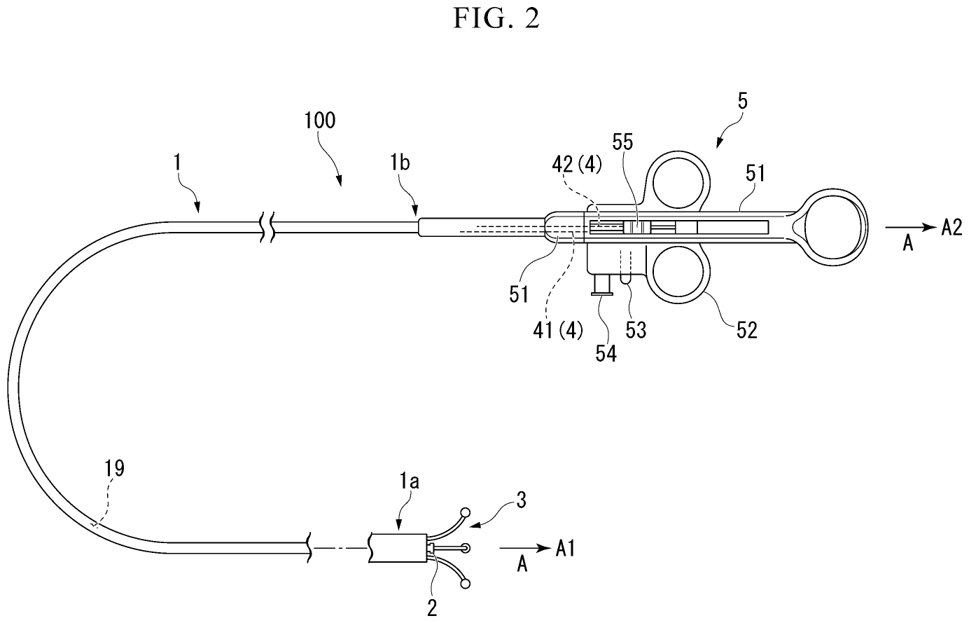

FIG. 2 is an overall view showing the treatment device 100. The treatment device (endoscopic treatment device) 100 includes a sheath 1, a rod 2, a support portion 3 (insulative support member), an operation wire 4, and an operation portion 5. The operation wire 4 includes a first operation wire 41 and a second operation wire 42. In the following description, in a longitudinal direction A of the treatment device 100, a side that is inserted into the body of a patient is referred to as a “tip-end side (distal-end side) A1”, and the operation portion 5 side is referred to as a “base-end side (proximal-end side) A2”.

The sheath 1 is an elongated resin member having flexibility and insulating properties and extending from a distal end 1a to a proximal end 1b. The sheath 1 has an internal space (conduit line, lumen) 19. The sheath 1 has an outer diameter suitable to be inserted into the channel 206 of the endoscope 200 and to be advanceable and retractable through the channel 206. As shown in FIG. 1, in a state in which the sheath 1 is inserted into the channel 206, the distal end 1a of the sheath 1 can protrude from and retract into the distal-end opening portion 206a of the channel 206.

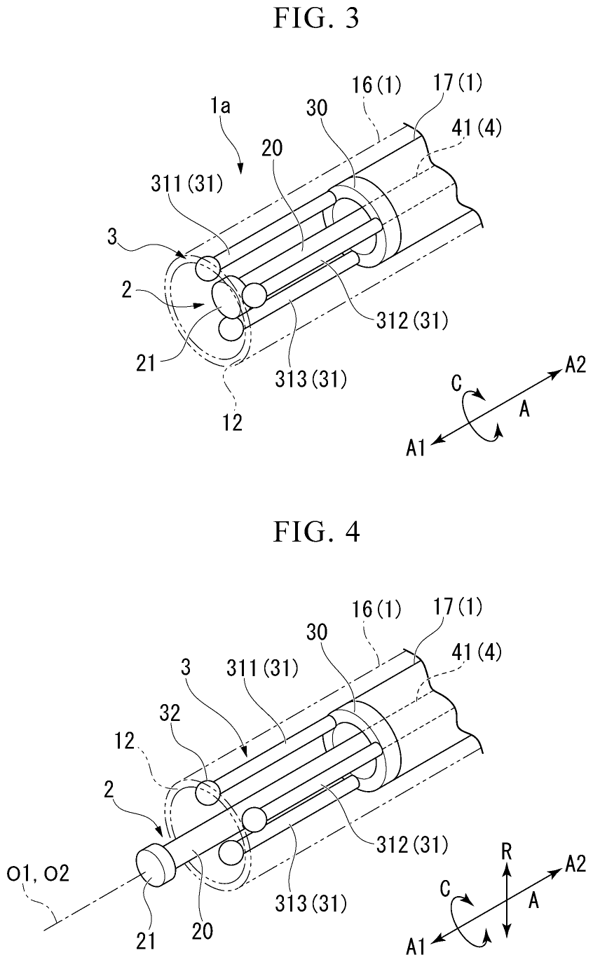

FIG. 3 is a perspective view showing the distal-end portion of the treatment device 100.

The sheath 1 includes an outer sheath (tube) 16 and an inner sheath 17 to be advanceable and retractable through the outer sheath 16. A distal-end opening 12 that opens in the longitudinal direction A is provided at the distal end of the outer sheath 16. The rod 2 and the support portion 3 are movable to protrude from the distal-end opening 12 and retractable into the distal-end opening 12.

The rod (electrode, knife) 2 is a substantially round bar-shaped member that is made of metal. The rod 2 is made of a material such as stainless steel or the like. The rod 2 has conductivity and is energized with a high-frequency current. The rod 2 is provided on the distal-end side A1 of the sheath 1. The rod 2 includes a rod main body 20 and a flange 21.

FIG. 4 is a cross-sectional view showing the distal-end portion of the treatment device 100 in which the rod 2 protrudes.

The rod 2 is movable protruding from the distal-end opening 12 and retracting into the distal-end opening 12 at the distal-end side A1. A central axis O2 in the longitudinal direction A of the rod 2 is substantially coincided with the central axis O1 in the longitudinal direction A of the sheath 1.

The rod main body 20 is a round bar-shaped member made of metal. The first operation wire 41 is attached to the proximal end of the rod main body 20. The high-frequency current is supplied to the rod main body 20 from the first operation wire 41 connected to the operation portion 5. When the high-frequency current is supplied from the first operation wire 41 to the rod 2, the rod main body 20 and the flange 21 function as monopolar electrodes that output the high-frequency current to the living tissue.

The flange (distal-end diameter-enlarged portion) 21 is a disk-shaped conductive member provided at the distal end of the rod main body 20. In the front view that is viewed along the longitudinal direction A, the outer circumference of the flange 21 is formed concentrically with the outer circumference of the rod main body 20. A length of the flange 21 in the radial direction R orthogonal to the longitudinal direction A is larger than a length of rod main body 20 in the radial direction R.

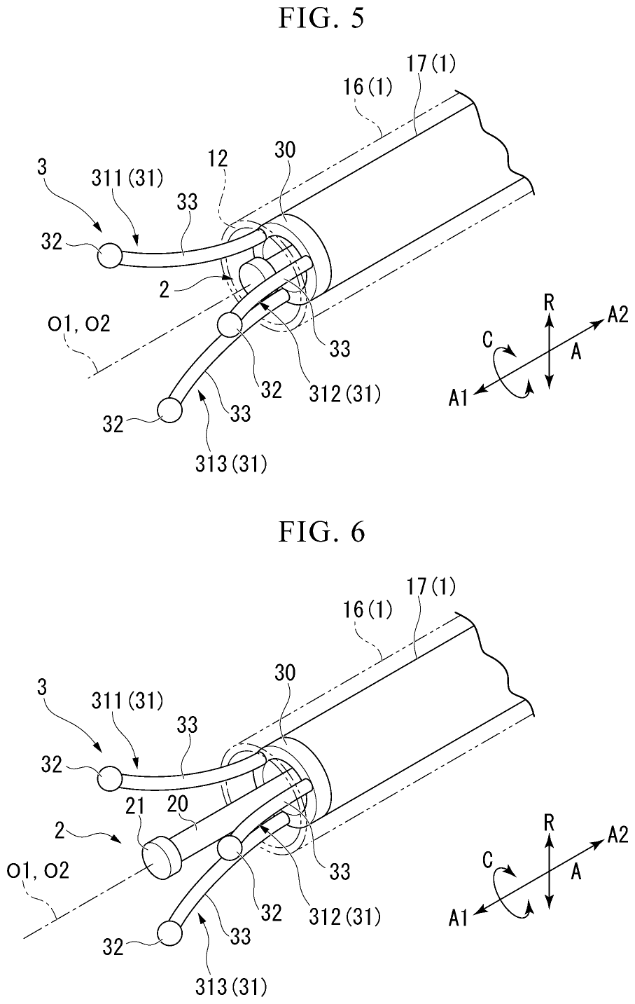

FIG. 5 is a cross-sectional view showing the distal-end portion of the treatment device 100 in which the support portion 3 protrudes.

The support portion 3 is a member capable of supporting the living tissues such as mucous membrane and the like. The support portion 3 is made of an insulating member such as resin or the like. Even if the support portion 3 contacts the rod 2, there is no high frequency current flowing through the support portion 3. The support part 3 can protrude from the distal-end opening 12 toward the distal-end side A1. The support portion 3 has a connection portion (proximal-end portion) 30 and forceps (support member) 31. The connection portion 30 is formed in an annular shape and attached to the distal end of the inner sheath 17.

The forceps (support member) 31 has a first forceps (first support body) 311, a second forceps (second support body) 312, and a third forceps (third support body) 313. The three forceps 31 are attached to the distal-end side A1 of the connection portion 30. The three forceps 31 are arranged outside the rod 2 in the radial direction R. The three forceps 31 are evenly arranged along the circumferential direction C with respect to the longitudinal direction A. It is noted that the number and arrangement of the forceps 31 are not limited to this configuration. The forceps (support member) 31 may have only one forceps 31 or only two forceps 31.

The forceps 31 have a distal-end portion 32 and a rod-shaped portion 33. The distal-end portion 32 is formed in a substantially spherical shape such that it is difficult for the distal-end portion 32 to damage the living tissues. The rod-shaped portion 33 is bent outward in the radial direction R on the tip-end side (distal-end side) A1. Therefore, as shown in FIG. 5, when the three forceps 31 protrude from the distal-end opening 12 of the sheath 1, the three forceps 31 expand (diameter enlargement) toward the tip-end side (distal-end side) A1.

In other words, when the forceps 31 protrudes from the distal end of the outer sheath 16, a first length is longer than a second length. The forceps 31 includes a first portion at a distal end of the forceps 31 and having the first length in a direction orthogonal to the longitudinal axis of the outer sheath 16. And forceps 31 includes a second portion at a proximal end of forceps 31 and having the second length in the direction orthogonal to the longitudinal axis of the outer sheath 16.

In other words, the first forceps 311 and the second forceps 312 are circumferentially separated from each other and are spaced apart radially from the rod 2. An imaginary circle or polygon in a plane perpendicular to the longitudinal axis and having a periphery intersecting the first forceps 311 and a second forceps 312 defines an interior area through which the rod 2 is movable to advance and retract.

FIG. 6 is a cross-sectional view showing the distal-end portion of the treatment device 100 in which the rod 2 and the support portion 3 protrude.

The rod 2 and the support portion 3 are independently operable. The rod 2 is advanceable and retractable along the longitudinal direction A among the first forceps 311, the second forceps 312, and the third forceps 313.

A maximum protrusion amount of the support portion 3 from the distal-end opening 12 of the outer sheath (tube) 16 may be larger than a maximum protrusion amount of the rod 2 from the distal-end opening 12. In this case, it is easy for the surgeon to visually confirm the flange 21 of the rod 2 without accommodating the support portion 3 in the outer sheath 16.

The maximum protrusion amount of the support portion 3 from the distal-end opening 12 of the outer sheath (tube) 16 may be smaller than the maximum protrusion amount of the rod 2 from the distal-end opening 12. In this case, it is possible to prevent any unintentionally contact of the rod 2 to the living tissue by protruding the support portion 3 to the distal-end side A1 of the flange 21 of the rod 2.

The operation wire 4 is a wire made of metal that is inserted through the internal space (conduit line, lumen) 19 of the sheath 1. For example, the operation wire 4 is formed of the material such as stainless steel or the like. The operation wire 4 includes a first operation wire 41 and a second operation wire 42.

The first operation wire 41 is a wire configured to operate the rod 2. A distal end of the first operation wire 41 is connected to the rod 2, and a proximal end of the first operation wire 41 is connected to a slider 52 of the operation portion 5.

The second operation wire 42 is a wire configured to operate the support portion 3. A distal end of the second operation wire 42 is connected to the inner sheath 17, and a proximal end of the second operation wire 42 is connected to a lever 55 of the operation wire 5. The distal end of the second operation wire 42 may be directly connected to the connection portion 30 of the support portion 3 without passing through the inner sheath 17. In this case, the inner sheath 17 is unnecessary to be provided.

As shown in FIG. 1 and FIG. 2, the operation portion 5 includes an operation portion main body 51, the slider 52, a power-supply connector 53, a fluid-supply port 54, and the lever (second slider) 55.

The distal-end portion of the operation portion main body 51 is connected to the proximal end 1b of the sheath 1. The operation portion main body 51 has an internal space for the operation wire 4 to be inserted through. The operation wire 4 passes through the internal space of the tube 10 and the internal space of the operation portion main body 51 to extend until the slider 52.

The slider 52 is attached to be movable along the longitudinal direction A with respect to the operation portion main body 51. The proximal-end portion of the first operation wire 41 is attached to the slider 52. The first operation wire 41 and the rod 2 advance and retract by the surgeon relatively advancing and retracting the slider 52 with respect to the operation portion main body 51.

The power-supply connector 53 is fixed to the slider 52. The power-supply connector 53 is connectable to a high-frequency power supply apparatus that is not shown in the figures, and the power-supply connector 53 is connected to the proximal-end portion of the first operation wire 41 via a conductive wire. The power-supply connector 53 is able to supply the high-frequency current supplied from the high-frequency power supply apparatus to the rod 2 via the first operation wire 41.

The fluid-supply port 54 is provided in the slider 52. The fluid-supply port 54 is connected to the internal space (conduit line, lumen) 19 of the sheath 1. The fluid supplied from the fluid-supply port 54 passes through the internal space (conduit line, lumen) 19 of the sheath 1 and discharged from the distal-end port 12.

The lever (second slider) 55 is attached to be relatively movable in the longitudinal direction A with respect to the slider 52. The proximal-end portion of the second operation wire 42 is attached to the lever 55. The second operation wire 42, the inner sheath 17, and the support portion 3 relatively advance and retract with respect to the first operation wire 41 and the rod 2 by the surgeon relatively advancing and retracting the lever 55 with respect to the slider 52.

### [Usage Method of Endoscopic Treatment System 300]

Next, the surgery procedures using the endoscopic treatment system 300 according to the present embodiment (usage method of the endoscopic treatment system 300) will be described. More specifically, the description will be made with regard to the incision and dissection treatment with respect to the lesion portion in the endoscopic treatment (endoscopic procedures) such as the ESD ((Endoscopic Submucosal Dissection) or the like.

As a preparation operation, the operator identifies the lesion portion by a known method. Specifically, the surgery inserts the insertion portion 202 of the endoscope 200 into the gastrointestinal tract (for example, the esophagus, the stomach, the duodenum, and the large intestine) to observe the image obtained by the imaging portion 203 of the endoscope 200 so as to identify the lesion portion. The surgeon locally injects a drug solution (for example, the physiological saline) into the submucosal layer SM of the lesion portion LE as the incision target as necessary.

### <Insertion Step>

The surgeon inserts the treatment device 100 into the channel 206 and protrudes the distal end 1a of the sheath 1 from the distal-end opening portion 206a of the insertion portion 202. The surgeon relatively advances the slider 52 of the operation portion 5 with respect to the operation portion main body 51 to protrude the rod 2 from the distal-end opening 12.

### <Incision Step>

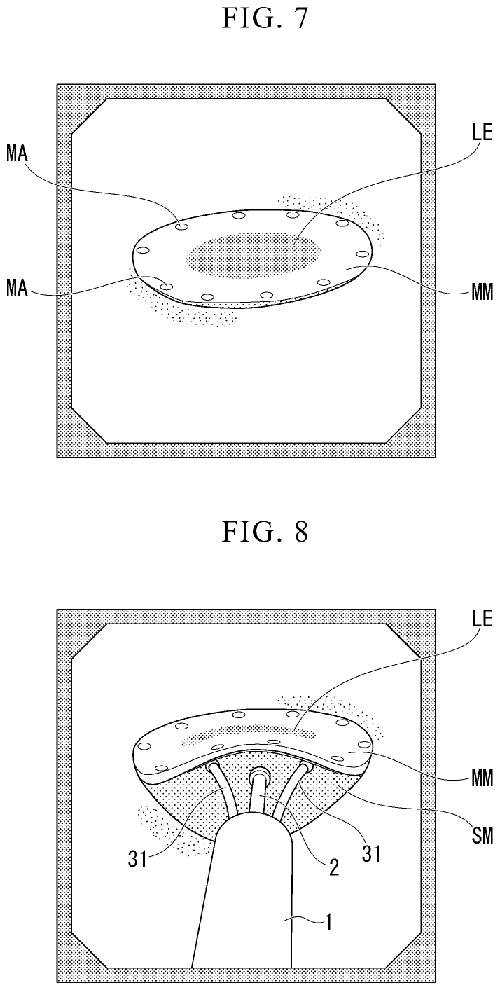

FIG. 7 is a view showing the incision step.

The surgeon advances the rod 2 to press the flange 21 to the mucous membrane MM in the vicinity of the lesion portion LE in a state in which the rod 2 is energized by the high-frequency current to apply a marking MA on the mucous membrane MM in the vicinity of the lesion portion LE. The surgeon moves the rod 2 in the state in which the rod 2 is energized by the high-frequency current to incise the mucous membrane MM in the vicinity of the lesion portion LE.

### <Dissection Step>

FIG. 8 is a view showing the dissection step.

The surgeon advances the rod 2 and the forceps 31 in the state of being energized by the high-frequency current to lift the incised flap-shaped mucous membrane MM so as to dissect the submucosa layer SM of the lesion portion LE while cauterizing the submucosa layer SM. It is possible for the surgeon to turn up the flap-shaped mucosa membrane MM that is incised by the forceps 31 to dissect the submucosa layer SM of the lesion portion LE by the rod 2 in a state of exposing the submucosa layer SM so as to perform the dissection step easily in a short period.

The surgeon continues the above-described operations (treatment) as necessary, and finally dissects the lesion portion LE is excised and finishes the ESD procedures.

It is easy to perform the treatment such as the dissection treatment or the like due to the endoscopic treatment system 300 according to the present embodiment.

As described above, the first embodiment of the present disclosure has been described in detail with reference to the drawings; however, the specific configuration is not limited to the present embodiment, and design changes and the like are included within the scope of the present disclosure. Also, the configurational elements shown in the present embodiment and the modification examples shown below can be combined as appropriate.

### Modification Example 1-1

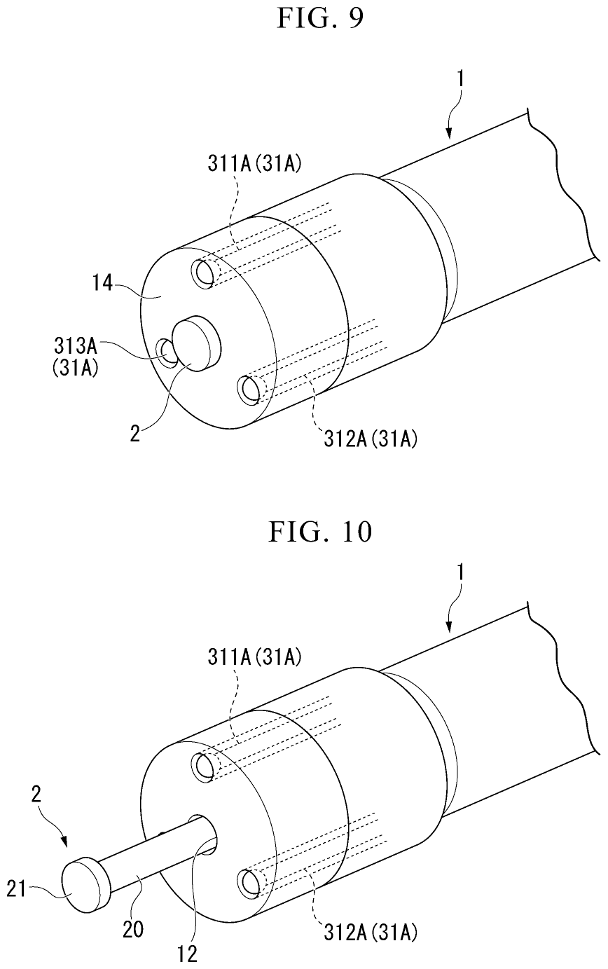

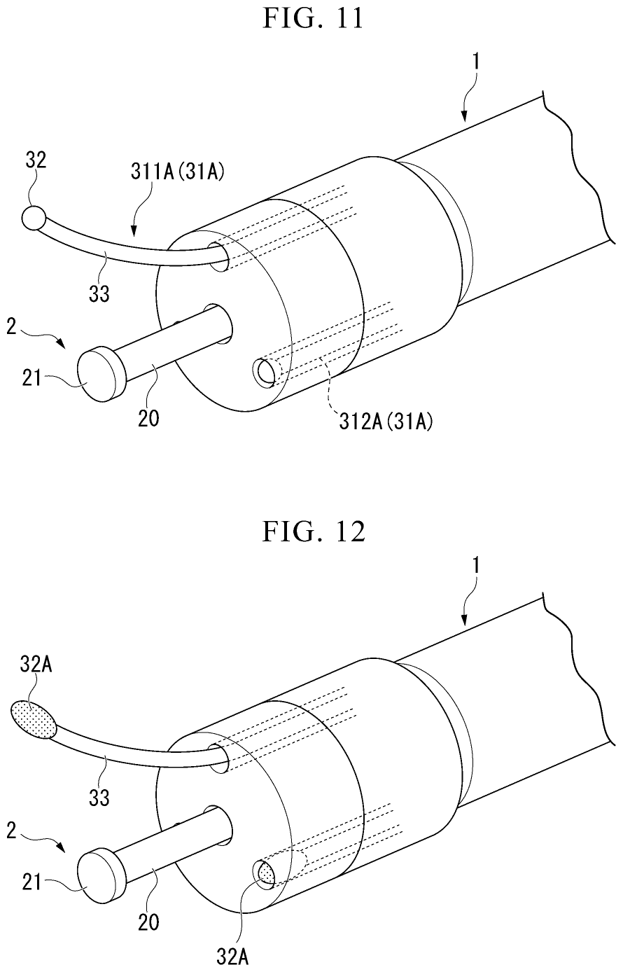

In the above-described embodiment, the three forceps 31 operate in conjunction. However, the aspect and operations of the forceps 31 are not limited to this configuration. FIG. 9 to FIG. 11 are perspective views showing a forceps 31A that is a modification example of the forceps 31. The forceps (support member) 31A has a first forceps (first support member) 311A, a second forceps (second support member) 312A, and a third forceps (third support member) 313A. The three forceps 31A are connected to different operation wires and can operate independently. In the dissection step, the surgeon can suitably turn up the flap-shaped mucosa membrane MM by protruding only necessary forceps 31A according to the state of the incised flap-shaped mucosa membrane MM.

### Modification Example 1-2

In the above-described embodiment, the distal-end portion 32 of the forceps 31 is formed in a substantially spherical shape. However, the aspect of the forceps 31 is not limited to this configuration. FIG. 12 is a perspective view showing a distal-end portion 32A that is a modification example of the distal-end portion 32. The surface of the distal-end portion 32A is made of rubber, and the distal-end portion 32A functions as an anti-slip member. FIG. 13 is a perspective view showing a distal-end portion 32B that is a modification example of the distal-end portion 32. The surface of the distal-end portion 32B is formed in a brush-shape, and the distal-end portion 32B functions as the anti-slip member.

### Modification Example 1-3

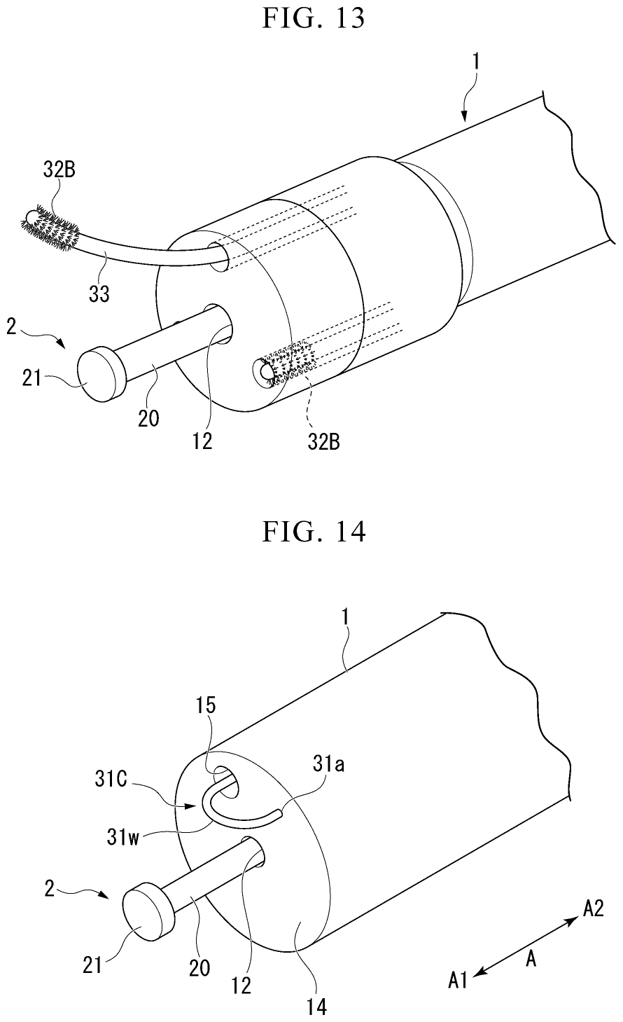

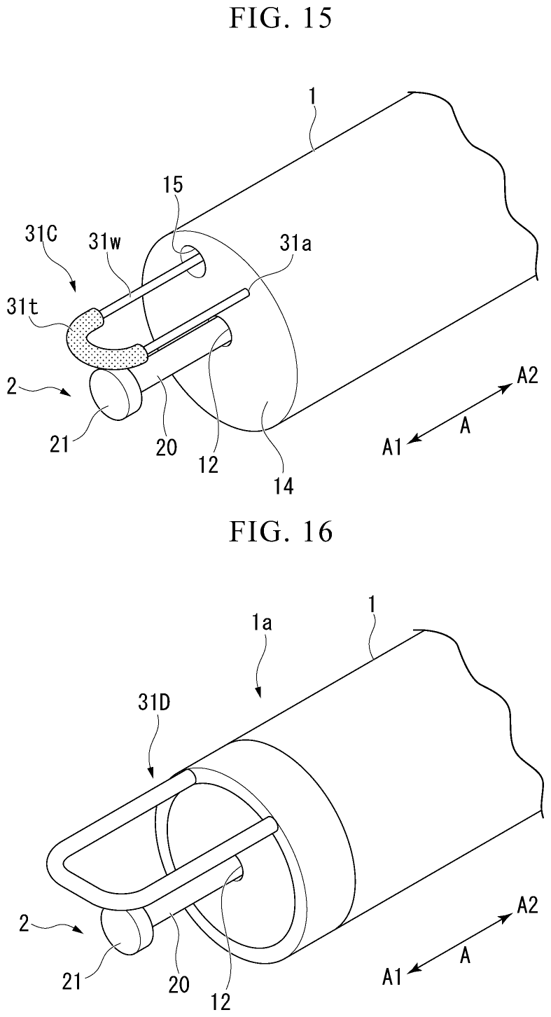

In the above-described embodiment, the forceps 31 can be completely accommodated in the sheath 1. However, the aspect of the forceps 31 is not limited to this configuration. FIG. 14 and FIG. 15 are perspective views showing a support member 31C that is a modification example of the forceps (support member) 31. The support member 31C includes a wire 31w and an anti-slip tube 31t. The wire 31w is inserted through a through hole 15 that is formed at another location with respect to the distal-end opening 12 on the distal-end surface 14 of the sheath 1, and the distal-end portion 31a is fixed to the distal-end surface 14 of the sheath 1. The anti-slip tube 31t is attached to the outer circumferential surface of the wire 31w which is the distal-end portion of the wire 31w. As shown in FIG. 15, the distal-end portion of the wire 31w becomes a loop shape by moving the wire 31 toward the distal-end side A1. The surgery can use the loop-shaped wire 31w and the anti-slip tube 31t to turn up the flap-shaped mucosa membrane MM. The portion where the through hole 15 is provide on the distal-end surface 14 of the sheath 1 may be formed in a tapered shape. In this case, the through hole 15 is provided at the proximal-end side A1 with respect to the distal-end opening 12. The wire 31w is not limited to a single wire and may be configured from a plurality of wires. The support member 31C is not limited to a single support member and may be configured in plural.

### Modification Example 1-4

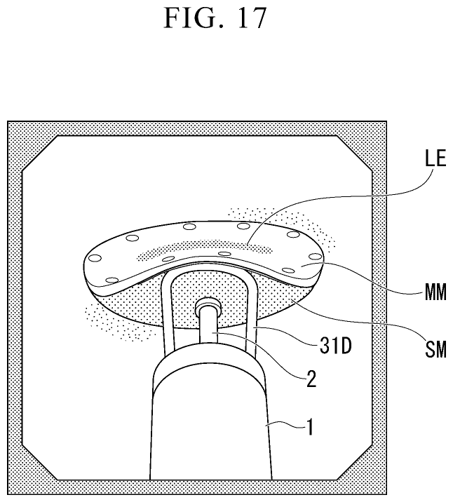

In the above-described embodiment, the forceps 31 is movable protruding from the sheath 1 and retractable into the sheath 1. However, the aspect of the forceps 31 is not limited to this configuration. FIG. 16 is a perspective view showing a forceps 31D that is a modification example of the forceps 31. The forceps 31D is made of metal or resin and formed in a U shape. The forceps 31D are fixed to the distal-end 1a of the sheath 1 to be unmovable with respect to the sheath 1. The protrusion amount of the sheath 31D from the distal-end opening 12 of the outer sheath (tube) 16 is larger than the maximum protrusion amount of the rod 2 from the distal-end opening 12. FIG. 17 is a view showing the dissection step using the forceps 31D. The surgeon can dissect the submucosa layer SM of the lesion portion by using the rod 2 in a state of turning up the flap-shaped mucosa membrane MM that is incised by the forceps 31D to expose the submucosa layer SM. The forceps 31D may be freely attachable to and detachable from the distal end 1a of the sheath 1. The forceps 31D may extend to be inclined outward in the radial direction of the rod 2. In this case, the distal end of the forceps 3D is positioned slightly outward in the radial direction than the outer diameter of the sheath 1.

### Modification Example 1-5

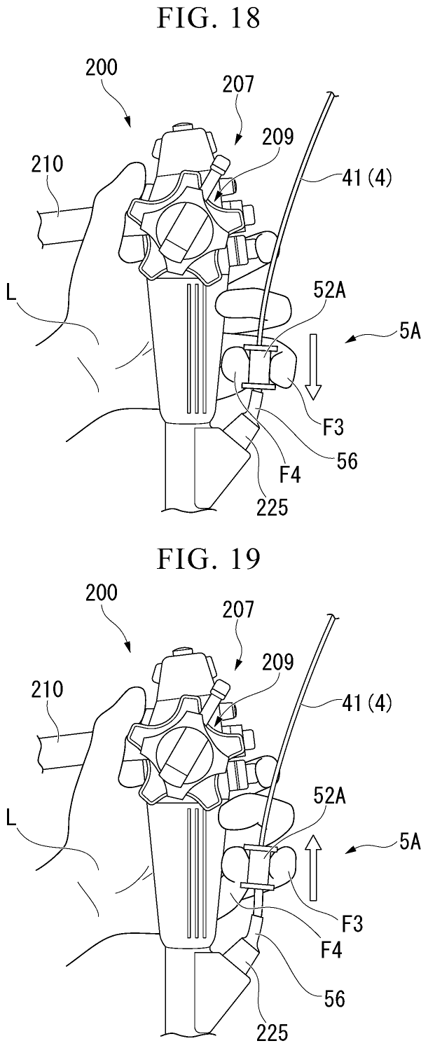

In the above-described embodiment, the rod 2 advances and retracts by the surgeon relatively advancing and retracting the slider 52 with respect to the operation portion main body 51. However, the aspect of the slider 52 of the operation portion 5 is not limited to this configuration. FIG. 18 and FIG. 19 are views showing an operation portion 5A that is a modification example of the operation portion 5. The operation portion 5A includes a slider 52A and a guide tube 56. The slider 52A is formed in a cylindrical shape and can be easily grasped by the ring finger F3 and the little finger F4 of the left hand L of the surgeon. The slider 52A is directly attached to the first operation wire 41 and arranged at a position where it can be easily grasped with the ring finger F3 and the little finger F4 of the left hand L of the surgeon holding the operation portion 207. The guide tube 56 is attached to the proximal-end opening (forceps opening) 206b in a state in which the first operation wire 41 is inserted therethrough. The guide tube 56 is configured to prevent the buckling of the first operation wire 41. It is possible for the surgeon to advance and retract the slider 52A while operating the operation portion 207 with the left hand L so as to advance and retract the rod 2 without using the right hand.

### Second Embodiment

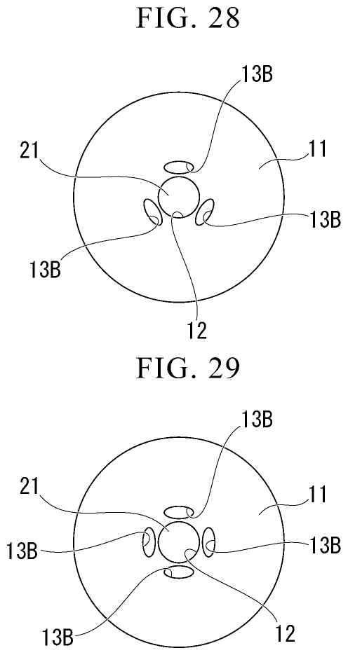

An endoscopic treatment system 300B according to a second embodiment of the present disclosure will be described with references from FIG. 20 to FIG. 30. In the following description, the described configurations and the common configurations will be designated with the same reference signs and the duplicate description will be omitted.

The endoscopic treatment system 300B includes the endoscope 200 and a treatment device 100B. The treatment device 100B is inserted into the endoscope 200 to be used.

FIG. 20 is an overall view showing the treatment device 100B.

The treatment device (endoscopic treatment, high-frequency treatment device) 100B includes a sheath 1B, the rod 2, an operation wire 4B, and an operation portion 5B.

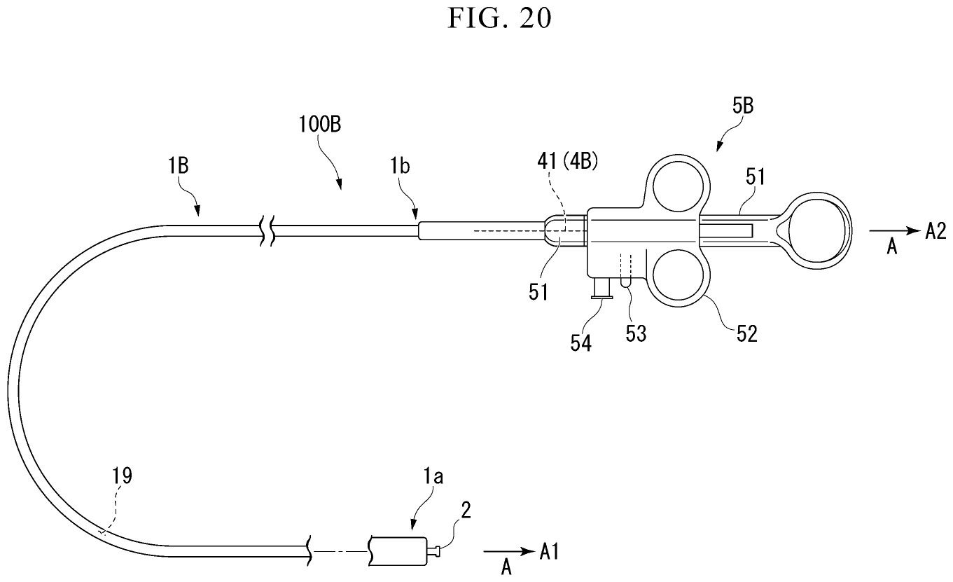

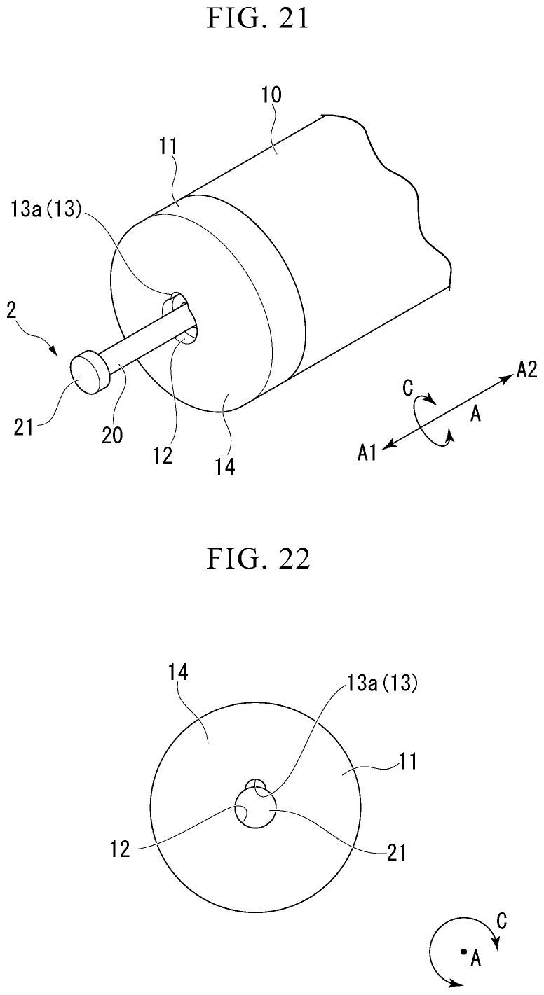

FIG. 21 is a perspective view showing a distal-end portion of the treatment device 100B.

The sheath 1B is an elongated tubular member extending from a distal end 1a to a proximal end 1b. The sheath 1B includes the internal space (conduit line, lumen) 19. The sheath 1B has an outer diameter suitable to be inserted into the channel 206 of the endoscope 200 and to be advanceable and retractable through the channel 206. The sheath 1B includes the tube 10 extending in the longitudinal direction A and a distal-end tip 11 provided at the distal end of the tube 10.

FIG. 22 is a front view showing the distal-end portion of the treatment device 100B.

The distal-end tip 11 is formed in a substantially cylindrical shape. A first through hole 12 and a second through hole 13 are formed in the distal-end tip 11. The first through hole (distal-end opening) 12 is a hole provided in the distal-end tip 11 and penetrating the distal-end tip 11 in the longitudinal direction A. The rod 2 is inserted through the first through hole 12. The second through hole 13 is a hole provided in the distal-end tip 11 and penetrating the distal-end tip 11 in the longitudinal direction A. In the present embodiment, the first through hole 12 and the second through hole 13 communicate with each other.

The distal end of the second through hole 13 communicates with the opening 13a formed on the distal-end surface 14 of the distal-end tip 11. The opening 13a formed at the distal end of the second through hole 13 opens toward the longitudinal direction A. The proximal end of the second through hole 13 communicates with the internal surface of the tube 10. The internal space of the tube 10 and the second through hole 13 form a conduit line 19 for supplying the fluid. The fluid suppled to the fluid-supply port 54 is discharged from the opening 13a at the distal-end side A1 of the second through hole 13 via the conduit line 19.

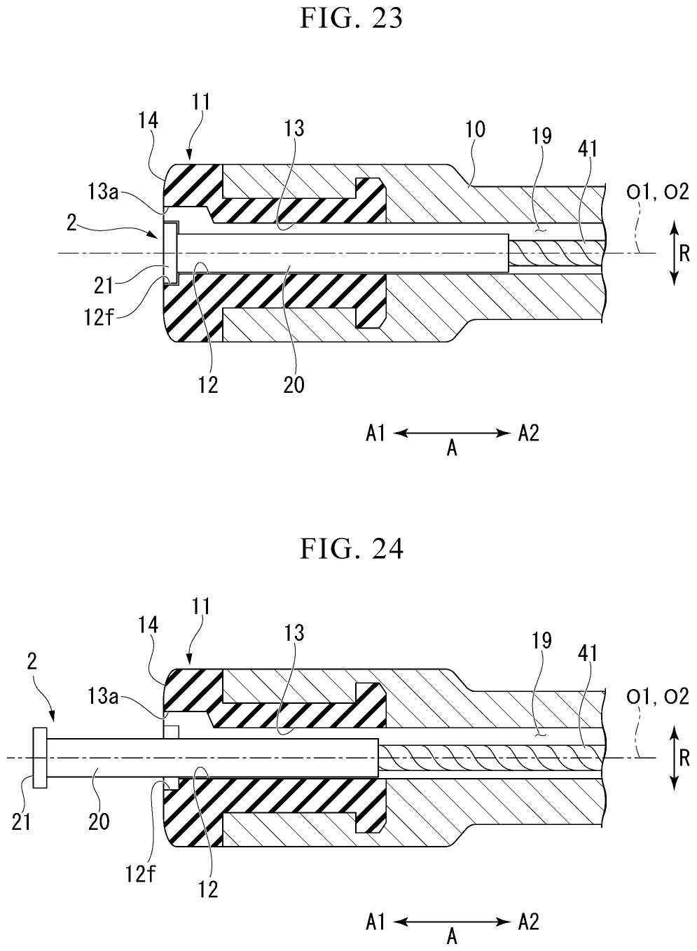

FIG. 23 is a cross-sectional view showing the distal-end portion of the treatment device 100B in which the rod 2 is accommodated.

A flange accommodation portion 12f capable of accommodating the flange 21 of the rod 2 is formed in the first through hole 12 of the distal-end tip 11. Even at the time when the rod 2 is accommodated in the sheath 1B and the flange 21 is accommodated in the flange accommodation portion 12f, the second through hole 12 communicates with the internal space of the tube 10.

FIG. 24 is a cross-sectional view showing the distal-end portion of the treatment device 100B in which the rod 2 is protruded.

The rod 2 is inserted through the first through hole 12 of the distal-end tip 11 of the sheath 1B along the longitudinal direction A, and the rod 2 is movable protruding from the first through hole 12 toward the distal-end side A1. The central axis O2 of the rod 2 in the longitudinal direction A is substantially coincided with the central axis O1 of the sheath 1B in the longitudinal direction A.

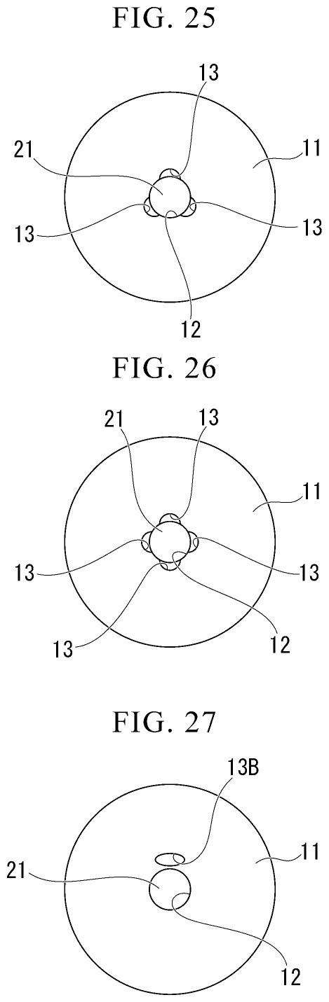

FIG. 25 and FIG. 26 are front views of the distal-end portion of the treatment device 100B according to another embodiment.

The second through hole 13 included in the distal-end tip 11 is not limited to one. The distal-end tip 11 may have three second through holes 13 as shown in FIG. 25, or have fourth second through holes 13 as shown in FIG. 26. The second through holes 13 as shown in FIG. 25 and FIG. 26 are evenly arranged along the circumferential direction C.

FIG. 27 to FIG. 29 are front views showing the distal-end portion of the treatment device 100B according to another embodiment.

The second through hole 13 may not communicate with the first through hole 12. The second through hole 13B that is a modification example of the second through hole 13 as shown in FIG. 27 to FIG. 29 are arranged outward in the radial direction R to be separated from the first through hole 12 and the second through holes 13B are evenly arranged along the circumferential direction C.

The operation wire 4B is a wire made of metal and configured to be inserted through the internal space (conduit line, lumen) 19 of the sheath 1B. The operation wire 4B is formed of the material such as the stainless steel, for example. The operation wire 4B includes the first operation wire 41.

As shown in FIG. 20, the operation portion 5B includes the operation portion main body 51, the slider 52, the power-supply connector 53, and the fluid-supply port 54.

### [Usage Method of Endoscopic Treatment System 300B]

Next, the surgery procedures using the endoscopic treatment system 300B according to the present embodiment (usage method of the endoscopic treatment system 300B) will be described. More specifically, the description will be made with regard to the incision and dissection treatment with respect to the lesion portion in the endoscopic treatment (endoscopic procedures) such as the ESD ((Endoscopic Submucosal Dissection) or the like.

As a preparation operation, the operator identifies the lesion portion by a known method. Specifically, the surgery inserts the insertion portion 202 of the endoscope 200 into the gastrointestinal tract (for example, the esophagus, the stomach, the duodenum, and the large intestine) to observe the image obtained by the imaging portion 203 of the endoscope 200 so as to identify the lesion portion. The surgeon locally injects a drug solution (for example, the physiological saline) into the submucosal layer SM of the lesion portion LE as the incision target as necessary.

### <Insertion Step>

The surgeon inserts the treatment device 100B into the channel 206 and protrudes the distal end 1a of the sheath 1B from the distal-end opening portion 206a of the insertion portion 202. The surgeon relatively advances the slider 52 of the operation portion 5B with respect to the operation portion main body 51 to protrude the rod 2 from the distal-end opening 12.

### <Incision Step>

The surgeon advances the rod 2 to press the flange 21 to the mucous membrane MM in the vicinity of the lesion portion LE in a state in which the rod 2 is energized by the high-frequency current to apply a marking MA on the mucous membrane MM in the vicinity of the lesion portion LE. The surgeon moves the rod 2 in the state in which the rod 2 is energized by the high-frequency current to incise the mucous membrane MM in the vicinity of the lesion portion LE.

### <Dissection Step>

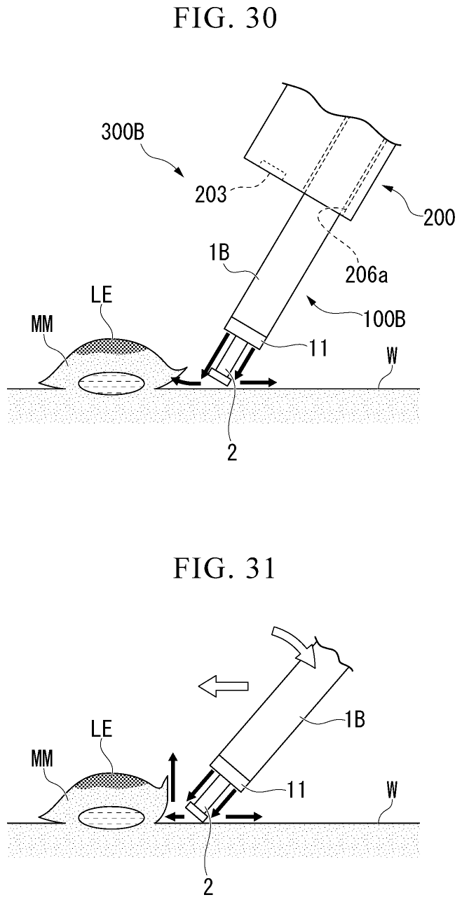

FIG. 30 to FIG. 33 are views showing the dissection step.

As shown in FIG. 30, the surgeon lifts the mucosa membrane MM by supplying a gas such as carbon dioxide to the fluid-supply port 54 and spraying the gas against the gastrointestinal wall W from the second through hole 13 to indirectly supply the gas to the lesion portion LE.

As shown in FIG. 31, the surgeon continues performing the gas supply to gradually make the distal end of the treatment device 100B to be inclined with respect to the gastrointestinal wall W while making the distal end thereof to approach the lesion portion LE to turn up the mucosa membrane MM.

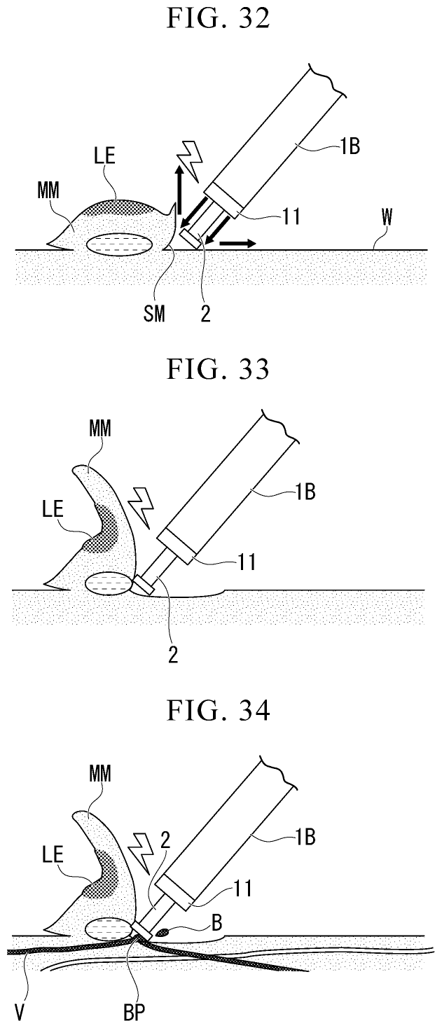

As shown in FIG. 32, the surgeon secures the state in which the dissection surface can be sufficiently visually confirmed and brings the rod 2 to contact the submucosa layer SM to dissect the submucosa layer SM of the lesion portion LE by the rod 2. At this time, the surgeon continues the gas supply to directly supply the gas to the submucosa layer SM.

As shown in FIG. 33, the surgeon dissects the submucosa layer SM. The surgeon finishes the gas supply when the state in which the mucosa membrane MM is turned up or the state in which the distal end of the endoscope 200 is sufficiently deeply slipped is reached. The surgeon can further dissect the submucosa layer SM of the lesion portion LE by the rod 2 in the state of turning up the flap-shaped mucosa membrane MM that is incised by the forceps 31 to expose the submucosa layer SM to perform the dissection step easily in a short period.

### <Hemostasis Step>

FIG. 34 to FIG. 37 are views showing the hemostasis step.

As shown in FIG. 34, at the time of incising or dissecting the lesion portion LE having blood vessels V in the mucosal layer, bleeding occurs due to damage to the blood vessel V. In the case when the bleeding occurs, the surgeon performs the hemostasis treatment.

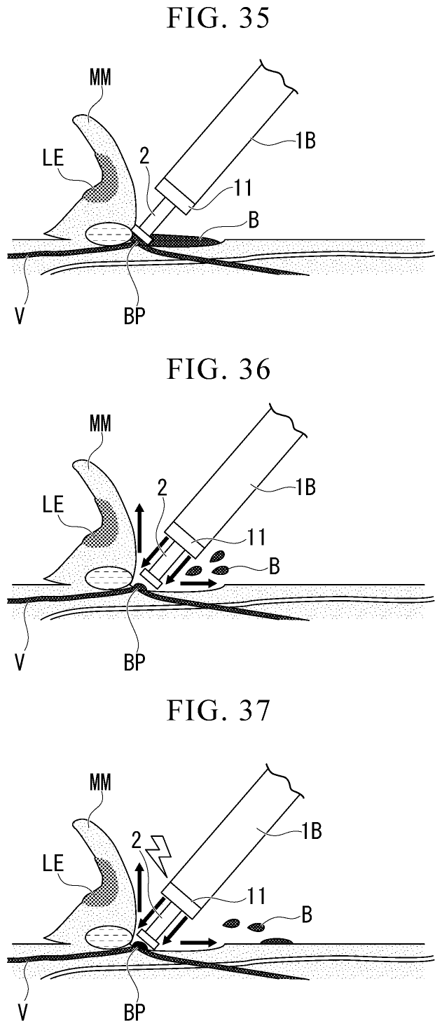

As shown in FIG. 35, blood B accumulates due to bleeding such that the bleeding point BP cannot be visually recognized. In addition, when the blood B accumulates, it becomes difficult for the surgeon to visually recognize the surgical field by the endoscope 200. Therefore, the blood B can be removed from the surgical field as necessary.

As shown in FIG. 36, the surgeon specifies the bleeding point BP by blowing off the accumulated blood B due to the gas supply. Even after specifying the bleeding point BP, the surgeon continues to supply the gas to maintain the visual recognition of the bleeding point BP.

As shown in FIG. 37, the surgeon applies current for coagulation in the state of brining the rod 2 to contact with the bleeding point BP to perform the hemostasis treatment at the bleeding point BP.

The surgeon can perform the hemostasis treatment while constantly supplying gas and visually recognizing the bleeding point BP. Also, the removal of the blood B by supplying the gas can remove the blood B in a wide range due to the forced convection so as to make it easy to identify the bleeding point BP. Also, since the blood B as an electrolyte is removed, it is possible for the rod 2 to effectively energize the bleeding point BP at the time of coagulating the bleeding point BP.

The surgeon continues the above-described operations (treatment) as necessary to finally dissect the lesion portion LE and finishes the procedures of ESD.

According to the endoscopic treatment system 300B according to the present embodiment, it is easy to perform the treatment such as the dissection treatment and the hemostasis treatment.

In the above description, in the dissection, the case where the mucous membrane MM is lifted by indirectly supplying the gas to the lesion portion LE in the dissection step has been described. However, in the dissection, the surgeon may lift by suppling a liquid such as a physiological saline to the fluid supply port 54 and spraying the liquid against the gastrointestinal wall W from the second through-hole 13 to indirectly supply the liquid to the lesion LE. The mucous membrane MM may be elevated by direct water delivery.

Also, in the above description, in the hemostasis step, the case where specifying the bleeding point BP by blowing off the blood B due to the air supplied has been described. However, in the hemostasis step, the surgeon may specify the bleeding point BP by blowing off the blood B by supplying the liquid. Furthermore, even after specifying the bleeding point BP, the surgeon may continue supplying the liquid to maintain the visual recognition of the bleeding point BP.

As described above, the second embodiment of the present disclosure has been described in detail with reference to the drawings; however, the specific configuration is not limited to the present embodiment, and design changes and the like are included within the scope of the present disclosure. Also, the configurational elements shown in the present embodiment and the modification examples shown below can be combined as appropriate.

### Modification Example 2-1

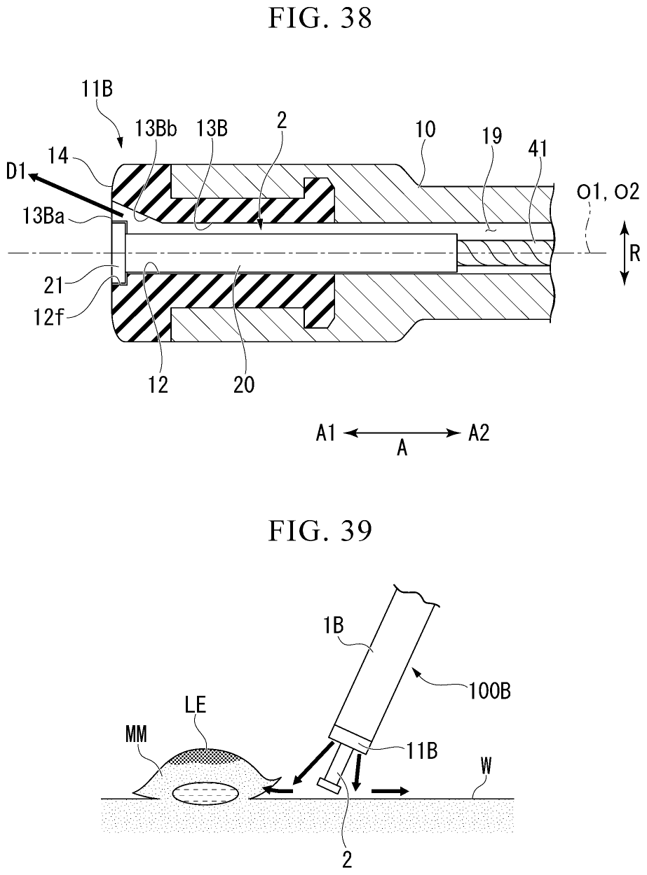

FIG. 38 is a cross-sectional view showing a distal-end tip 11B that is a modification example of the distal-end tip 11. A second through hole 13B that is a modification example of the second through hole 13 is formed in the distal-end tip 11B. The distal end of the second through hole 13B communicates with the opening 13Ba formed in the distal-end surface 14 of the distal-end tip 11B. The opening 13Ba formed at the distal end of the second through hole 13B opens outward in the radial direction with respect to the rod 2. The distal-end portion 13Bb of the second through hole 13B extends in a direction D1 intersecting with the longitudinal direction A. The direction D1 is a direction separating from the central axis O1 as toward the distal-end side A1 from the proximal-end side A2.

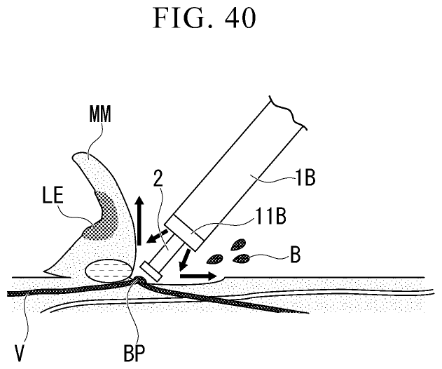

FIG. 39 is a step showing the dissection step. The gas supplied to the second through hole 13B of the distal-end tip 11B is supplied relative to the gastrointestinal wall W so as to be spread outward in the radial direction R from the opening 13Ba. Since the range of the gas supply is wide, it is suitable to perform the dissection step with respect to a wide lesion portion LE. FIG. 40 is a view showing the hemostasis step. Since the range of the gas supply is wide, it is suitable to blow off the blood B even in a case in which the bleed accumulation extends to a wider range.

Also, a liquid such as physiological saline may be supplied to the second through hole 13B of the distal tip 11B and may be supplied relative to the gastrointestinal wall W so as to spread outward in the radial direction R from the opening 13Ba.

### Third Embodiment

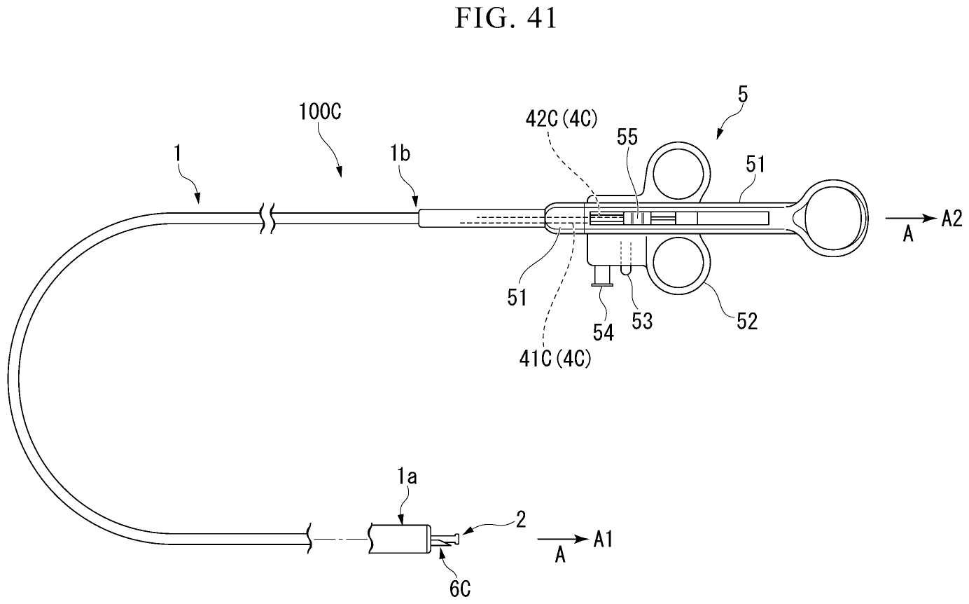

An endoscopic treatment system 300C according to a third embodiment of the present disclosure will be described with references from FIG. 41 to FIG. 48. In the following description, the described configurations and the common configurations will be designated with the same reference signs and the duplicate description will be omitted.

The endoscopic treatment system 300C includes the endoscope 200 and a treatment device 100C. The treatment device 100C is inserted into the endoscope 200 to be used.

FIG. 41 is an overall view showing the treatment device 100C.

The treatment device (endoscopic treatment device) 100C includes the sheath 1, a rod 2C, a sharp member (hollow needle) 6C, an operation wire 4C, and the operation portion 5. The operation wire 4C includes a first operation wire 41C and a second operation wire 42C.

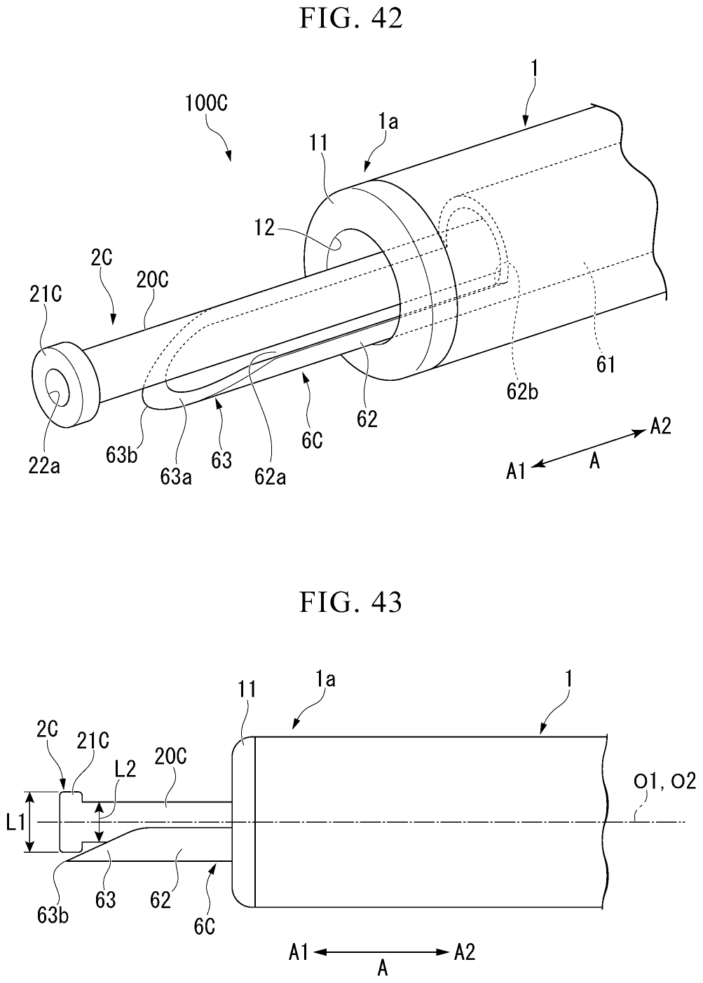

FIG. 42 is a perspective view showing a distal-end portion of the treatment device 100C.

The distal-end tip 11 having the through hole 12 penetrating in the longitudinal direction A is attached to the distal end 1a of the sheath 1. The rod 2C and the sharp member 6C are inserted into the through hole 12.

FIG. 43 is a side view showing the distal-end portion of the treatment device 100C.

The rod 2C is a substantially round bar-shaped member that is made of metal, and the rod 2C is provided to be freely movable to protrude from the through hole 12 of the distal-end tip 11 of the sheath 1 toward the distal-end side A1. The rod 2C is formed of the material such as the stainless steel or the like, for example. The rod 2C has the conductivity and is energized by the high-frequency current. The rod 2C includes a rod main body 20C and a flange 21C.

The rod 2C is inserted through the sharp member 6C along the longitudinal direction A and is relatively movable with respect to the sharp member 6C. The central axis O2 of the rod 2C in the longitudinal direction A is substantially coincided with the central axis O1 of the sheath 1 in the longitudinal direction A.

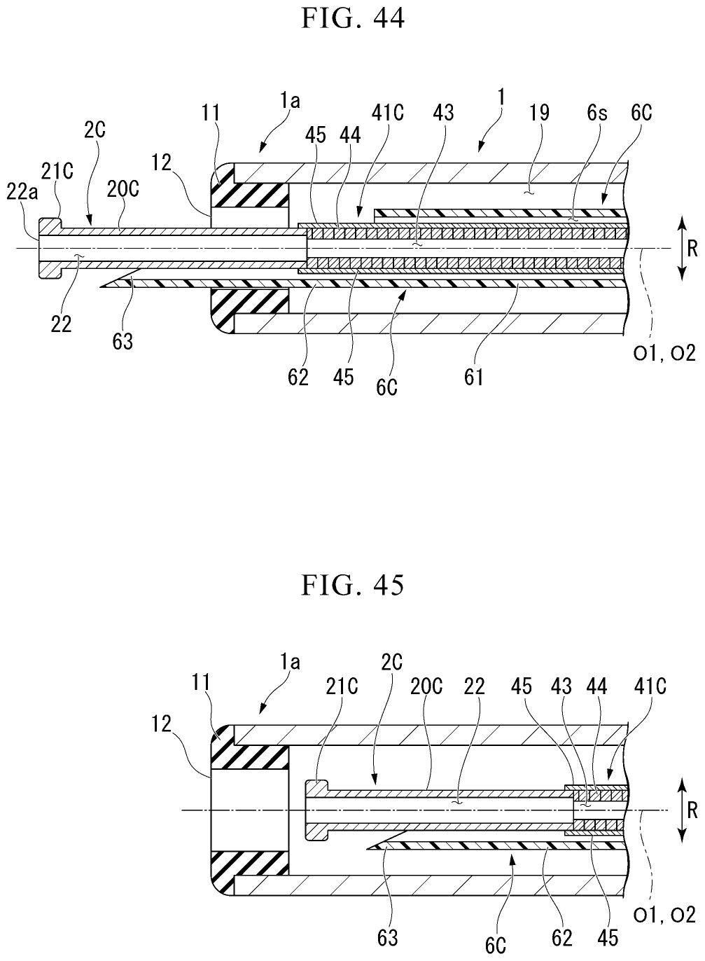

FIG. 44 is a cross-sectional view showing the distal-end portion of the treatment device 100C.

The rod main body 20C is a round bar-shaped member that is made of metal. The first operation wire 41C is attached to the proximal end of the rod main body 20C. The rod main body 20C supplies the high-frequency current that is supplied from the first operation wire 41C connected with the operation portion 5 to the flange 21C. When the high-frequency current is supplied from the first operation wire 41C to the rod 2C, the rod main body 20C and the flange 21C function as the monopolar electrode to output the high-frequency current to the living tissue.

The flange 21C is a disk-shaped conductive member provided at the distal end of the rod main body 20C. In the front view viewed along the longitudinal direction A, the outer circumference of the flange 21C is formed concentrically with the outer circumference of the rod main body 20C. As shown in FIG. 43, the length L1 of the flange 21C in the radial direction R orthogonal to the longitudinal direction A is larger than the length L2 of the rod main body 20C in the radial direction R.

The rod main body 20C and the flange 21C include a first water-supply conduit line 22 extending along the longitudinal direction A. The first water-supply conduit line 22 communicates with the distal-end opening 22a formed in the flange 21C. The distal-end opening 22a opens at the distal-end side A1.

The sharp member (hollow needle) 6C is a tubular member made of resin material, metal material, or the like. The rod 2C and the first operation wire 41C are inserted in an internal space 6s of the sharp member 6C so as to be advanceable and retractable therethrough. The sharp member 6C includes a tubular main body portion 61, a distal-end portion 62 and a sharp portion 63.

The tubular main body portion 61 is a cylindrical member, and the proximal end thereof is connected to the second operation wire 42C. The distal-end portion 62 is provided at the distal end of the tubular main body portion 61.

The distal-end portion 62 is provided at the distal end of the tubular main body portion 61 and is formed in a semi-cylindrical shape by dividing the cylindrical member along the longitudinal direction A. That is, the distal-end portion 62 includes a slit-formation portion 62a formed in the semi-cylindrical shape. A part of the flange 21C protrudes outward in the radial direction R from the outer circumferential surface and/or the inner circumferential surface of the tubular main body portion 61, and the flange 21C is configured to be freely slidable along the inner circumferential surface at the distal-end side A1 than the proximal end 62b of the slit-formation portion 62a. Also, the slit-formation portion 62a does not have to be formed in the semi-cylindrical shape, and for example, the slit-formation portion 62a may be a configuration that is formed as a groove formed along the longitudinal axis to have a width smaller than the inner diameter of the tubular main body portion 61. In this case, the flange 21C is formed to have the dimension suitable for entering the groove such that the flange 21C is slidable inside the groove.

The sharp portion 63 is a member in which the distal-end side A1 thereof provided at the distal end of the distal-end portion 62 is sharp. As shown in FIG. 42 and FIG. 43, the sharp member 63 is formed in a shape in which a semi-cylindrical-shaped edge 63a at the distal-end side A1 is inclined with respect to the longitudinal direction A. The distal end 63b of the sharp portion 63 is pointed toward the distal-end side A1.

FIG. 45 is a cross-sectional view showing the distal-end portion of the treatment device 100C.

The rod 2C and the sharp member 6C are retracted so as to be able to be accommodated at the proximal-end side A2 from the through hole 12 of the distal-end tip 11 of the sheath 1.

The operation wire 4C is a wire made of metal that is inserted through the internal space (conduit line, lumen) 19 of the sheath 1. The operation wire 4C includes the first operation wire 41C and the second operation wire 42C.

The first operation wire 41C is a wire configured to operate the rod 2C. The first operation wire 41C is a shaft being inserted through the internal space 6s of the sharp member 6C, and includes a coil shaft 44 and a tube 45. The distal end of the first operation wire 41C is connected to the rod 2C, and the proximal end of the first operation wire 41C is connected to the slider 52 of the operation portion 5. Also, the first operation wire 41C only has to be a hollow shaft and may be configured in other aspect.

The coil shaft 44 is a coil wire made of metal. The coil shaft 44 is formed of the material such as the stainless steel or the like, for example. Inside the coil shaft 44, the second water-supply conduit line 43 is formed. The second water-supply conduit line 43 is connected to the proximal end of the first water-supply conduit line 22. The fluid supplied from the fluid-supply port 54 is discharged from the distal-end opening 22a through the second water-supply conduit line 43 and the first water-supply conduit line 22.

The tube 45 is a tube provided at the outer circumferential portion of the coil shaft 44, and is a heat-shrinkable tube, for example. The liquid is not leaked from the second water-supply conduit line by covering the tube 45 on the outer circumferential portion of the coil shaft 44.

The second operation wire 42C is a wire configured to operate the sharp member 6C. The distal end of the second operation wire 42C is connected to the sharp member 6C, and the proximal end of the second operation wire 42C is connected to the lever 55 of the operation portion 5.

### [Usage Method of Endoscopic Treatment System 300C]

Next, the surgery procedures using the endoscopic treatment system 300C according to the present embodiment (usage method of the endoscopic treatment system 300C) will be described. More specifically, the description will be made with regard to the incision and dissection treatment with respect to the lesion portion in the endoscopic treatment (endoscopic procedures) such as the ESD ((Endoscopic Submucosal Dissection) or the like.

As a preparation operation, the operator identifies the lesion portion by a known method. Specifically, the surgery inserts the insertion portion 202 of the endoscope 200 into the gastrointestinal tract (for example, the esophagus, the stomach, the duodenum, and the large intestine) to observe the image obtained by the imaging portion 203 of the endoscope 200 so as to identify the lesion portion.

### <Insertion Step>

The surgeon inserts the treatment device 100C into the channel 206 and protrudes the distal end 1a of the sheath 1B from the distal-end opening portion 206a of the insertion portion 202. The surgeon relatively advances the slider 52 of the operation portion 5 with respect to the operation portion main body 51 to protrude the rod 2 and the sharp member 6.

### <Local-Injection Step>

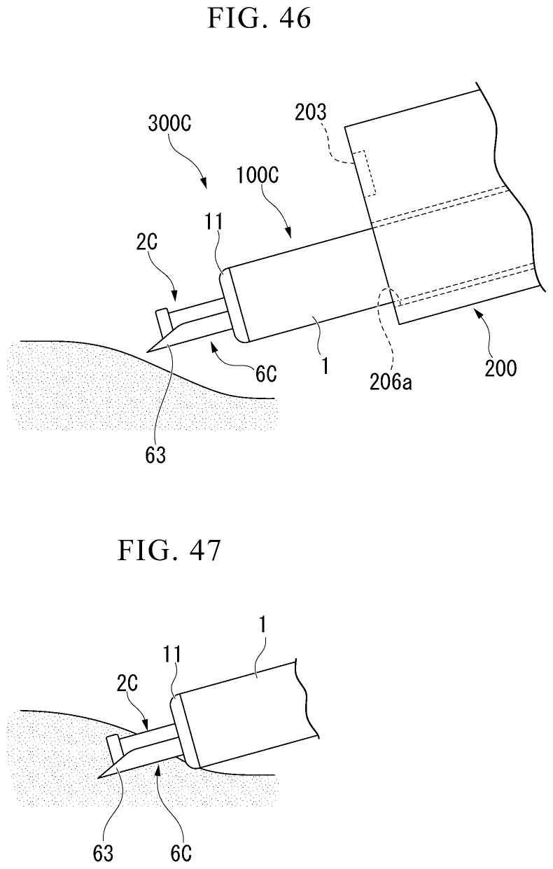

FIG. 46 to FIG. 48 are view showing the local-injection step.

As shown in FIG. 46, the surgeon moves the slider 52 to the distal-end side A1 with respect to the lever 55 so as to move the sharp member 6C to the distal-end side A1 with respect to the rod 2C. As a result, the sharp portion 63 of the sharp member 6C is protruded to the distal-end side A1 than the distal end of the rod 2C. Also, at this time, part of the flange 21C is in a state of being covered by part of the sharp member 6C, and the remaining part of the flange 21C is exposed from the part of the sharp member 6C. Also, at this time, it is unnecessary for the rod 2C to protrude from the distal-end tip 11 to the maximum extent, the flange 21C may be in contact with the distal-end tip 11, and the flange 21C may be positioned at the proximal-end side A2 than the distal-end tip 11.

As shown in FIG. 47, the surgeon punctures the sharp portion 63 of the sharp member 6C to penetrate the portion in the lesion portion where the liquid for the local injection (local-injection liquid) is injected. More specifically, the surgeon makes a cut from the surface of the mucosal membrane to the submucosal layer with the tip (needle tip) 63b of the sharp portion 63.

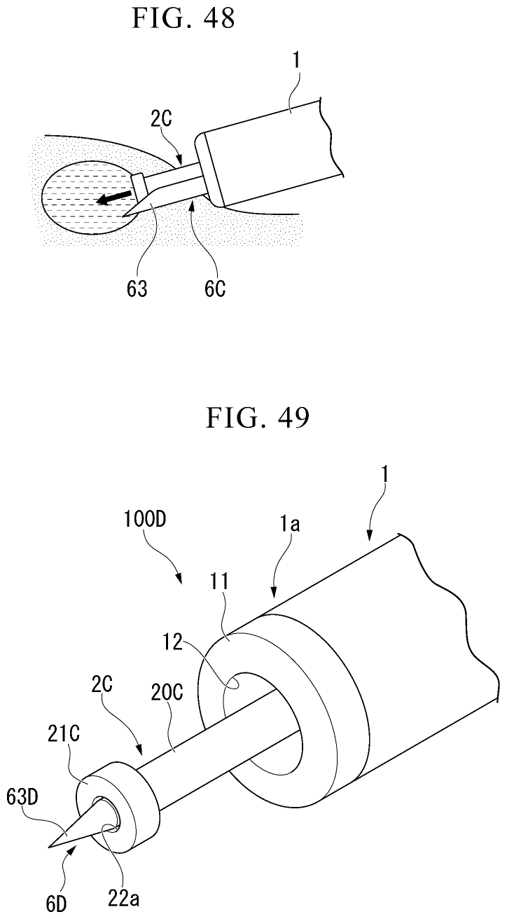

As shown in FIG. 48, in a state in which the distal-end opening 22a of the distal end of the rod 2C is inserted into the submucosa layer, the surgeon supplies the drug solution (for example, the physiological saline) with respect to the fluid-supply port 54, and supplies the water from the distal-end opening 22a (local-injection step). More specifically, in a state of puncturing the sharp portion 63 of the sharp member 6C into the cut, the surgeon supplies the drug solution from the distal-end opening 22a toward the cut. The surgeon bulges the surface of the mucous membrane by injecting the drug solution from the distal-end opening 22a through the cut into the submucosal layer. The surgeon may supply the drug solution from the distal-end opening 22a toward the cut while pressing the distal end of the rod 2C against the cut.

According to the local-injection step by using the treatment device 100C, it is possible to perform the initial local injection with respect to the portion where the mucosa membrane is thick. Also, it is possible to form a circular bulge similar to the method of using a local-injection needle. Also, it is easy to adjust the size of the bulged portion. Also, it is possible to perform the initial local injection using a syringe by attaching the syringe to the liquid-supply port 54.

### <Incision-Dissection Step>

The surgeon performs the incision-dissection step. The surgeon advances the rod 2C to move the flange 21 in the state of being energized with the high-frequency current to incise the mucosa membrane in the vicinity of the lesion portion. Also, the surgeon advances the rod 2C in the state of being energized with the high-frequency current to lift the incised mucosa membrane of the lesion portion to expose the submucosa layer while dissecting the submucosa layer of the incised lesion portion. Similar to the second embodiment, the surgeon supplies the gas such as the carbon dioxide with respect to the liquid-supply port 54 to blow the gas against the gastrointestinal wall W from the distal-end opening 22a to lift the mucous membrane MM. During the incision-dissection treatment, the sharp member 6C can be completely accommodated in the sheath 1.

### <Hemostasis Step>

In a case in which the bleeding occurs during the incision-dissection treatment, the surgeon performs the hemostasis treatment. The surgeon presses the rod main body 20C and the flange 21C thereon while cauterizing the bleeding point with the energization by the high-frequency current to stop the bleeding (hemostasis step). Similar to the second embodiment, the surgeon supplies the gas such as the carbon dioxide or the like to the liquid-supply port 54 and blows the gas from the distal-end opening 22a to the gastrointestinal wall W to blow off the blood B to specify the bleeding point BP.

The surgeon continues the above-described operations (treatment) as necessary to finally dissect the lesion portion LE and finishes the procedures of ESD.

According to the endoscopic treatment system 300C according to the present embodiment, it is easy to perform the treatment such as the dissection treatment and the hemostasis treatment.

As described above, the third embodiment of the present disclosure has been described in detail with reference to the drawings; however, the specific configuration is not limited to the present embodiment, and design changes and the like are included within the scope of the present disclosure. Also, the configurational elements shown in the present embodiment and the modification examples shown below can be combined as appropriate.

### Fourth Embodiment

An endoscopic treatment system 300D according to a fourth embodiment of the present disclosure will be described with references from FIG. 49 to FIG. 53. In the following description, the described configurations and the common configurations will be designated with the same reference signs and the duplicate description will be omitted.

The endoscopic treatment system 300D includes the endoscope 200 and a treatment device 100D. The treatment device 100D is inserted into the endoscope 200 to be used.

FIG. 49 is a perspective view showing the distal-end portion of the treatment device 100D.

The treatment device (endoscopic treatment device) 100D includes the sheath 1, a rod 2C, a sharp member 6D, the operation wire 4C, and the operation portion 5.

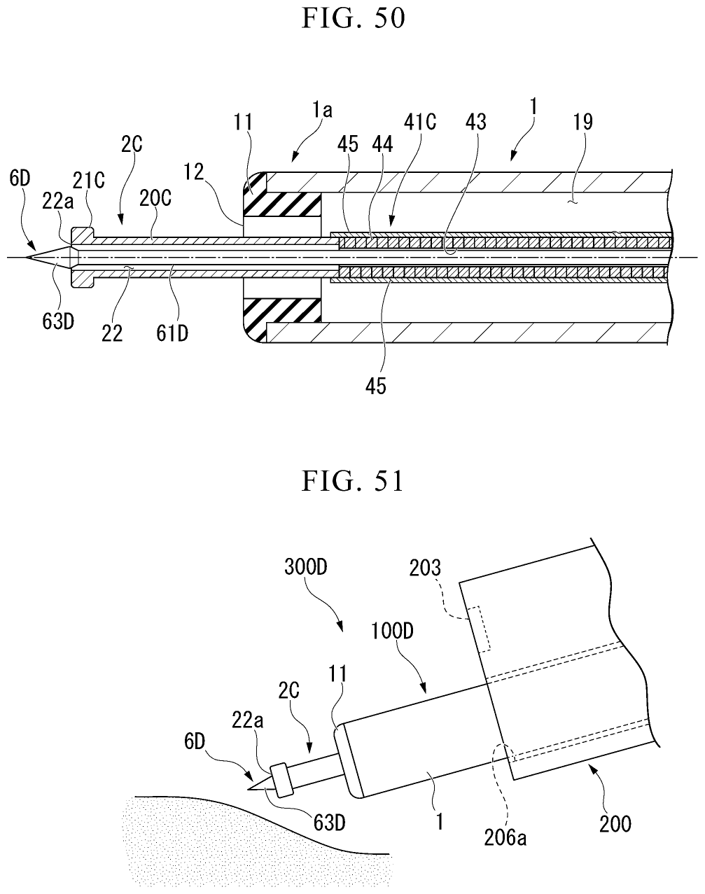

FIG. 50 is a cross-sectional view showing the distal-end portion of the treatment device 100D.

The sharp member 6D is a rod-shaped member formed of the resin material or the metal material or the like. The sharp member 6D is inserted through the first water-supply conduit line of the rod 2C to be advanceable and retractable therethrough. The sharp member 5D is freely movable to protrude from the distal-end opening 22a of the rod 2C. The conduit line for supplying the fluid is formed between the outer circumferential surface of the sharp member 6D and the inner circumferential surface of the first water-supply conduit line 22 of the rod 2C. The sharp member 6D includes a main body portion 61D and a sharp portion 63D provided at the distal end of the main body portion 61D.

The second operation wire 42C is a wire configured to operate the sharp member 6D. The distal end of the second operation wire 42C is connected to the sharp member 6D, and the proximal end of the second operation wire 42C is connected to the lever 55 of the operation portion 5.

### [Usage Method of Endoscopic Treatment System 300D]

Next, the surgery procedures using the endoscopic treatment system 300D according to the present embodiment (usage method of the endoscopic treatment system 300D) will be described. More specifically, the description will be made with regard to the incision and dissection treatment with respect to the lesion portion in the endoscopic treatment (endoscopic procedures) such as the ESD ((Endoscopic Submucosal Dissection) or the like.

As a preparation operation, the operator identifies the lesion portion by a known method. Specifically, the surgery inserts the insertion portion 202 of the endoscope 200 into the gastrointestinal tract (for example, the esophagus, the stomach, the duodenum, and the large intestine) to observe the image obtained by the imaging portion 203 of the endoscope 200 so as to identify the lesion portion.

### <Insertion Step>

The surgeon inserts the treatment device 100D into the channel 206 and protrudes the distal end 1a of the sheath 1B from the distal-end opening portion 206a of the insertion portion 202. The surgeon relatively advances the slider 52 of the operation portion 5 with respect to the operation portion main body 51 to protrude the rod 2C and the sharp member 6D.

### <Local-Injection Step>

FIG. 51 to FIG. 53 are view showing the local-injection step.

As shown in FIG. 51, the surgeon moves the lever 55 toward the distal-end side A1 with respect to the slider 52 so as to move the sharp member 6D toward the distal-end side A1 with respect to the rod 2C. As a result, the sharp portion 63D of the sharp member 6D is protruded toward the distal-end side A1 with respect to the rod 2C.

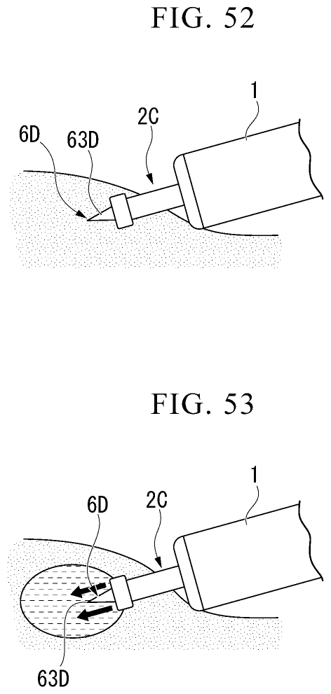

As shown in FIG. 52, the surgeon punctures the sharp portion 63D of the sharp member 6D to penetrate the portion in the lesion portion where the liquid for the local injection (local-injection liquid) is injected.

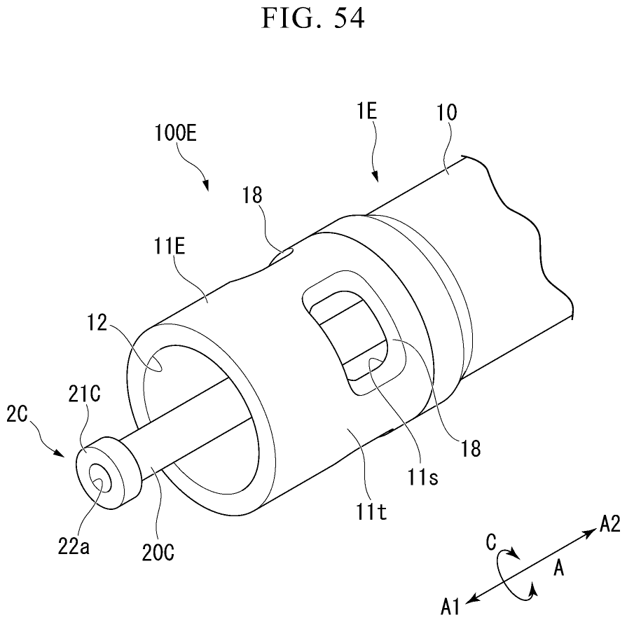

As shown in FIG. 53, in a state in which the distal-end opening 22a of the distal end of the rod 2C is inserted into the submucosa layer, the surgeon supplies the drug solution (for example, the physiological saline) with respect to the fluid-supply port 54, and supplies the water from the distal-end opening 22a (local-injection step).

According to the local-injection step by using the treatment device 100D, it is possible to perform the initial local injection with respect to the portion where the mucosa membrane is thick. Also, it is possible to form a circular bulge similar to the method of using a local-injection needle. Also, it is easy to adjust the size of the bulged portion. Also, it is possible to perform the initial local injection using a syringe by attaching the syringe to the liquid-supply port 54. Also, even in a case in which the living tissue enters the distal-end opening 22a and the first water-supply conduit line 22 of the rod 2C, it is possible to discharge the living tissue to the outside by the sharp member 6D.

### <Incision-Dissection Step>

The surgeon performs the incision-dissection step. The surgeon advances the rod 2C to move the flange 21 in the state of being energized with the high-frequency current to incise the mucosa membrane in the vicinity of the lesion portion. Also, the surgeon advances the rod 2C in the state of being energized with the high-frequency current to lift the incised mucosa membrane of the lesion portion to expose the submucosa layer while dissecting the submucosa layer of the incised lesion portion. Similar to the second embodiment, the surgeon supplies the gas such as the carbon dioxide with respect to the liquid-supply port 54 to blow the gas against the gastrointestinal wall W from the distal-end opening 22a to lift the mucous membrane MM. During the incision-dissection treatment, the sharp member 6D can be completely accommodated in the sheath 1.

### <Hemostasis Step>

In a case in which the bleeding occurs during the incision-dissection treatment, the surgeon performs the hemostasis treatment. The surgeon presses the rod main body 20C and the flange 21C thereon while cauterizing the bleeding point with the energization by the high-frequency current to stop the bleeding (hemostasis step). Similar to the second embodiment, the surgeon supplies the gas such as the carbon dioxide or the like to the liquid-supply port 54 and blows the gas from the distal-end opening 22a to the gastrointestinal wall W to blow off the blood B to specify the bleeding point BP.

The surgeon continues the above-described operations (treatment) as necessary to finally dissect the lesion portion LE and finishes the procedures of ESD.

According to the endoscopic treatment system 300D according to the present embodiment, it is easy to perform the treatment such as the dissection treatment and the hemostasis treatment.

As described above, the fourth embodiment of the present disclosure has been described in detail with reference to the drawings; however, the specific configuration is not limited to the present embodiment, and design changes and the like are included within the scope of the present disclosure. Also, the configurational elements shown in the present embodiment and the modification examples shown above can be combined as appropriate.

### Fifth Embodiment

An endoscopic treatment system 300E according to a fifth embodiment of the present disclosure will be described with reference to FIG. 54. In the following description, the described configurations and the common configurations will be designated with the same reference signs and the duplicate description will be omitted.

The endoscopic treatment system 300E includes the endoscope 200 and a treatment device 100E. The treatment device 100E is inserted into the endoscope 200 to be used.

FIG. 54 is a perspective view showing the distal-end portion of the treatment device 100E.

The treatment device (endoscopic treatment device) 100E includes a sheath 1E, the rod 2C, the first operation wire 4C, and the operation portion 5B.

The sheath 1E is an elongated resin member having flexibility and insulating properties and extending from a distal end 1a to a proximal end 1b. The sheath 1E has the internal space (conduit line, lumen) 19. The sheath 1E has an outer diameter suitable to be inserted into the channel 206 of the endoscope 200 and to be advanceable and retractable through the channel 206. The sheath 1E includes the tube 10 extending in the longitudinal direction A and a distal-end tip 11E provided at the distal end of the tube 10.

The distal-end tip 11E is formed in a substantially cylindrical shape. The first through hole 12 and a lateral hole 18 are formed in the distal-end tip 11E. The lateral hole 18 is a hole provided in the distal-end tip 11E and penetrating in the radial direction R that is orthogonal to the longitudinal direction A. The lateral hole 18 is a hole penetrating from the outer circumferential surface lit to the inner circumferential surface 11s of the distal-end tip 11E. The three lateral holes 18 are evenly provided along the circumferential direction C. Also, the number and the aspect of the lateral hole 18 is not limited to this configuration.

Similar to the third embodiment and the fourth embodiment, the surgeon uses the treatment device 100E to blow the gas from the distal-end opening 22a to the gastrointestinal wall W to lift the mucosa membrane MM. Also, the surgeon identifies the bleeding point BP by blowing the gas from the distal-end opening 22a to the gastrointestinal wall W to blow off the blood B. Furthermore, the surgeon can perform the suctioning from the first through hole 12 and the lateral hole 18 to suction the blood B from the surgery field.

According to the endoscopic treatment system 300E according to the present embodiment, it is easy to perform the treatment such as the dissection treatment and the hemostasis treatment.

As described above, the fifth embodiment of the present disclosure has been described in detail with reference to the drawings; however, the specific configuration is not limited to the present embodiment, and design changes and the like are included within the scope of the present disclosure. Also, the configurational elements shown in the present embodiment and the modification examples shown above can be combined as appropriate.

What is claimed is:

1. An endoscopic treatment device, comprising:
a hollow tube;
an electrode; and
an insulative support member,
wherein, in a radial direction relative to a longitudinal axis of the hollow tube, the insulative support member is radially outward of the electrode and the hollow tube is radially outward of the insulative support member.

2. The endoscopic treatment device according to claim 1, wherein the insulative support member is movable to protrude from and retract into a distal end of the hollow tube.

3. The endoscopic treatment device according to claim 2, wherein the insulative support member includes a first portion at a distal end of the insulative support member and having a first length in a direction orthogonal to the longitudinal axis of the hollow tube,
wherein the insulative support member includes a second portion at a proximal end of the insulative support member and having a second length in the direction orthogonal to the longitudinal axis of the hollow tube, and
wherein, when the insulative support member protrudes from the distal end of the hollow tube, the first length is longer than the second length.

4. The endoscopic treatment device according to claim 1, further comprising:
a first wire configured to flow a high-frequency current, and
a second wire connected to a proximal end of the insulative support member,
wherein the electrode is connected to a distal end of the first wire,
wherein the first wire is movable to be advanced and retracted in the hollow tube, and
wherein the second wire is movable to be advanced and retracted relative to the first wire.

5. The endoscopic treatment device according to claim 1, wherein the insulative support member includes a first support body and a second support body,
wherein the first support body and the second support body are located at a distal end of the insulative support member,
wherein the first support body and a second support body are circumferentially separated from each other and are spaced apart radially from the electrode, and
wherein an imaginary circle or polygon in a plane perpendicular to the longitudinal axis and having a periphery intersecting the first support body and a second support body defines an interior area through which the electrode is movable to advance and retract.

6. The endoscopic treatment device according to claim 2, wherein a maximum distance the insulative support member protrudes from the distal end of the hollow tube is larger than a maximum distance the electrode protrudes from the distal end of the hollow tube.

7. The endoscopic treatment device according to claim 2, wherein a maximum distance the insulative support member protrudes from the distal end of the hollow tube is smaller than a maximum distance the electrode protrudes from the distal end of the hollow tube.

8. The endoscopic treatment device according to claim 1, wherein the insulative support member is formed of a resin.

9. An endoscopic treatment device, comprising:
a hollow tube;
an electrode; and
an insulative support member,
wherein, in a radial direction relative to a longitudinal axis of the hollow tube, the insulative support member is radially outward of the electrode, and
wherein the insulative support member is movable relative to the electrode in a direction of the longitudinal axis of the hollow tube.

10. The endoscopic treatment device according to claim 9, wherein the insulative support member is movable to protrude from and retract into a distal end of the hollow tube.

11. The endoscopic treatment device according to claim 10, wherein the insulative support member includes a first portion at a distal end of the insulative support member and having a first length in a direction orthogonal to the longitudinal axis of the hollow tube,
wherein the insulative support member includes a second portion at a proximal end of the insulative support member and having a second length in the direction orthogonal to the longitudinal axis of the hollow tube, and
wherein, when the insulative support member protrudes from the distal end of the hollow tube, the first length is longer than the second length.

12. The endoscopic treatment device according to claim 9, further comprising:
a first wire configured to flow a high-frequency current, and
a second wire connected to a proximal end of the insulative support member,
wherein the electrode is connected to a distal end of the first wire,
wherein the first wire is movable to be advanced and retracted in the hollow tube, and
wherein the second wire is movable to be advanced and retracted relative to the first wire.

13. The endoscopic treatment device according to claim 9, wherein the insulative support member includes a first support body and a second support body,
wherein the first support body and the second support body are located at a distal end of the insulative support member,
wherein the first support body and a second support body are circumferentially separated from each other and are spaced apart radially from the electrode, and
wherein an imaginary circle or polygon in a plane perpendicular to the longitudinal axis and having a periphery intersecting the first support body and a second support body defines an interior area through which the electrode is movable to advance and retract.

14. The endoscopic treatment device according to claim 10, wherein a maximum distance the insulative support member protrudes from the distal end of the hollow tube is larger than a maximum distance the electrode protrudes from the distal end of the hollow tube.

15. The endoscopic treatment device according to claim 10, wherein a maximum distance the insulative support member protrudes from the distal end of the hollow tube is smaller than a maximum distance the electrode protrudes from the distal end of the hollow tube.

16. An endoscopic treatment method, comprising:
spraying a fluid from a treatment tool onto a mucous membrane; and
while spraying the fluid from the treatment tool:
moving the treatment tool toward a lesion portion of the mucous membrane to turn up the mucous membrane, and
incising a submucosa layer.

17. The endoscopic treatment method according to claim 16, further comprising removing blood from a surgical field by discharging the fluid from the treatment tool.

18. The endoscopic treatment method according to claim 16, wherein the fluid is a gas.

19. The endoscopic treatment method according to claim 16, wherein the fluid is a liquid.

20. An endoscopic treatment method, comprising:
spraying a fluid from the endoscopic treatment device according to claim 1 onto a mucous membrane; and
while spraying the fluid from the endoscopic treatment device:
moving the endoscopic treatment device toward a lesion portion of the mucous membrane to turn up the mucous membrane, and
incising a submucosa layer.
